(19) United States
(12) Patent Application Publication
(10) Pub. No.: US 2023/0081896 A1
(43) Pub. Date: Mar. 16, 2023

(54) SYSTEMS AND METHOD OF INTEGRATED AIR QUALITY MONITORING

(71) Applicant: The Regents of the University of Michigan, Ann Arbor, MI (US)

(72) Inventors: Byunghoon Ryu, Ann Arbor, MI (US); Jay Chen, Ann Arbor, MI (US); Xiaogan Liang, Ann Arbor, MI (US); Katsuo Kurabayashi, Ann Arbor, MI (US); Young Geun Park, Ann Arbor, MI (US)

(21) Appl. No.: 17/945,558

(22) Filed: Sep. 15, 2022

Related U.S. Application Data
(60) Provisional application No. 63/244,788, filed on Sep. 16, 2021.

(51) Int. Cl. G01N 33/569; G01N 21/59

(52) U.S. Cl. CPC G01N 33/56983; G01N 21/59; G01N 33/56916; G01N 2201/127; G01N 2333/165; G01N 2333/245

(57) ABSTRACT

Provided herein is an air monitoring system with a venturi pump including an air supply passageway, a sample passageway, and a discharge passageway, the discharge passageway in fluid communication with the air supply passageway and the sample passageway, and a detection device including a biochip, a light emitting source, a photodetector, and a controller electronically coupled to the photodetector. Also provided herein is a photonic biogel and uses thereof for spectroscopic detection of airborne pathogens.

### CROSS-REFERENCE TO RELATED APPLICATIONS

This application claims priority to U.S. Provisional Patent Application No. 63/244,788, filed on Sep. 16, 2021, the entire contents of which are incorporated herein by reference.

### STATEMENT REGARDING FEDERALLY-SPONSORED RESEARCH

The invention was made with Government support under CBET 2030551 awarded by the National Science Foundation. The Government has certain rights in the invention.

### BACKGROUND

The exposure of airborne particles such as viral and bacterial particles poses a serious health concern in our society, causing a variety of infections, severe respiratory diseases, problems in breathing, and allergies. Conventional airborne particle monitoring has been conducted on remotely collected air samples in an off-site centralized laboratory. In the conventional approach, sample analysis is usually performed with aqueous samples. As such, the first step of conventional airborne particle analysis requires the collection and transfer of airborne particles from the air to water by natural sediment or machine sampling. The machine sampling method involves large-scale machine-oriented sedimentation, percussion, centrifugal impingement, filtering, electrostatic attachment, and cyclonic separation followed by analysis using dynamic image analysis, static laser light scattering, laser diffraction, dynamic light scattering, and sieve analysis. Despite advances, fast sampling and accurate on-site detection of airborne particles is still a difficult technical task, especially in micro-climates.

The implementation of the existing approaches prevents direct collection and detection of airborne particles in the gas phase. Thus, conventional analysis techniques cannot ensure that the collected airborne particles specimen reflects the original state and cannot be directly used in sample analysis.

In addition, the acute respiratory coronavirus disease (COVID-19) has spread rapidly across the world after the outbreak of severe acute respiratory syndrome coronavirus 2 (SARS-CoV-2) in December 2019, causing death, illness, disruption of everyday life, and economic losses of businesses and individuals. The World Health Organization identified the COVID-19 outbreak as a pandemic on Mar. 12, 2020, as the human-to-human transmission rapidly increased. Since the outbreak to Dec. 1, 2020, more than 61.2 million cases of COVID-19 have been confirmed worldwide, which led to approximately 1.44 million deaths. As the estimated basic reproduction number of COVID-19 is approximately 2.2, on average, each patient spreads the infection to 2.2 people. Since specialized COVID-19 medications and vaccines are not yet available early diagnosis and management are necessary for the epidemic to be managed. The rapid spread of the COVID-19 pandemic reflects the shortcomings of the existing laboratory-based viral diagnostic testing model.

SARS-CoV-2 is an enveloped virus with large positive-sense single-stranded ribonucleic acid (RNA) genomes. SARS-CoV-2 has a single-positive strand RNA genome encoding four structural proteins: spike (S), envelope (E), matrix (M), and nucleocapsid (N). The fundamental limitations of current diagnostic assays for viral pathogens are related to their reliance on RNA genome analyses such as the polymerase chain reaction (PCR) analysis. This approach requires mainly temperature management as well as labor-intensive laboratory-based protocols for viral particle isolation, lysis, and removal of inhibiting materials. Although some methods include reverse transcription PCR and loop-mediated isothermal amplification technique to provide detection of SARS-CoV-2 (bypassing the need for RNA isolation/purification starting from a saliva sample or temperature cycling) the gene analysis technique is not easily adaptable for point-of-care (POC) airborne detection, because of the need for sample preparation, reaction control, sensitive equipment, and detection mechanism requiring aqueous phase solutions. Although conventional gene analysis techniques have been used as standard methods for clinical diagnostics, other sufficiently low cost and rapid approaches are required to provide diagnosis at the POC, particularly in air quality control.

The infection mechanism of the viral pathogenesis of SARS-CoV-2 has been investigated, and studies report that SARS-CoV-2 utilizes angiotensin-converting enzyme II (ACE2) as a cellular entry receptor, which is also a well-known host cell receptor for SARS-CoV. SARS-CoV-2 colocalizes with ACE2 in animal cells. Its spike (S-) protein binds ACE2 with a high affinity. The S-protein has been recognized as a molecular signature of SARS-CoV-2. Therefore, an S-protein analysis can indicate the SARS-CoV-2 infection by this process.

In the USA, as of October 2021, there have been over 44 million confirmed cases and 0.72 million reported deaths from the virus. Available evidence suggests that this virus spreads rapidly and is large-scale within and across communities. The United States Centers for Disease Control and Prevention (CDC) has acknowledged that breathing in or touching the eyes, nose, and mouth with small virus-contained droplets and particles on the surface of one's hands is one of the most common ways of getting infected by SARS-CoV-2. Aerosols or droplet nuclei can form airborne particles containing fungi, pollen, bacteria, or viruses. In early 2003, the World Health Organization (WHO) reported that aerosol transmission was responsible for a super spreading event of SARS in a housing block located in Hong Kong, China. The WHO report identified that the virus aerosols were transported in the wastewater plumbing system in the building and then spread through the empty U-bends in the bathrooms. Furthermore, recent studies have shown that SARS-CoV-2 remains viable in aerosols for at least three hours with limited reduction in infectious titers.

The fundamental limitations of the current gold standard assay for COVID-19 diagnosis originate from the polymerase chain reaction (PCR) analysis. This approach requires temperature management as well as labor-intensive laboratory protocols for viral particle isolation, lysis, and removal of inhibiting materials. Several methods have recently been developed to eliminate the need for RNA isolation/purification from a sample or temperature cycling, including nano-photothermal polymerase chain reaction (PCR), clustered regularly interspaced short palindromic repeats (CRISPR) machinery, and loop-mediated isothermal amplification (LAMP). However, these methods still require other forms of sample preparation, high-temperature (>50° C.) reaction control, handling of aqueous-phase solutions, and sensitive equipment operations. As such, these methods are not easily adaptable to detect the transmission pathways of airborne viral particles.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

One aspect of the present disclosure provides an airborne particle monitoring system with high sensitivity, fast speed, and simple operating capabilities for a micro-climate setting.

The rapid identification of COVID-19 airborne particles opens up an entirely new way to effectively prevent coronavirus infections. In-situ monitoring of virus particles in the air enables time-efficient and low-cost infection management. An integrated air quality monitoring system detailed herein allows for real-time detection of airborne severe acute respiratory syndrome coronavirus 2 (SARS-CoV-2) with high sensitivity. The detection unit of the system comprises a light-emitting diode, a biochip, a photodetector, and an air handler. The biochip incorporates biologically functionalized gold nanoparticles (AuNPs) embedded in a hydrogel layer, which serve as plasmonic nanoprobes detecting SARS-CoV-2 particles. An optical transmission change of the hydrogel layer induced by nanoprobes-virus interactions allows us to quantify SARS-CoV-2 in an air sample. The photocurrent variation (ΔI/I₀) of the photodetector resulting from the optical transmission change is directly correlated with the virus particle population. Furthermore, an application software detailed herein analyzes and wirelessly transmits airborne particle data in real time via Bluetooth communication, for example. In some embodiments, the photocurrent signal reaches a plateau within 5 min for SARS-CoV-2 and bacteria in the population range of 10⁻⁵˜10⁻¹ PFU/μL and 10³˜10⁷ CFU/mL, respectively.

One aspect of the present disclosure provides an air monitoring system having a venturi pump including an air supply passageway, a sample passageway, and a discharge passageway. The discharge passageway in fluid communication with the air supply passageway and the sample passageway. The system further includes a detection device including a biochip, a light emitting source, a photodetector, and a controller electronically coupled to the photodetector.

In some embodiments, the air supply passageway includes a first portion with a first diameter and a second portion with a second diameter, the second diameter is smaller than the first diameter.

In some embodiments, a ratio of the second diameter to the first diameter is 0.2.

In some embodiments, the second portion is positioned within the discharge passageway.

In some embodiments, the discharge passageway includes a cylindrical portion, a first tapered portion, and a second tapered portion, wherein the first tapered portion is positioned between the cylindrical portion and the second tapered portion.

In some embodiments, the sample passageway is oriented at an angle with respect to the air supply passageway.

In some embodiments, the system further includes a pressure sensor coupled to the sample passageway.

In some embodiments, the biochip includes an inlet, an outlet, and a trapping chamber positioned between the inlet and the outlet.

In some embodiments, the trapping chamber is rectangular and includes a length and a depth, and wherein a ratio of the length to the depth is within a range of 1 to 10.

In some embodiments, the trapping chamber is cylindrical and includes a diameter is equal to a light source diameter.

In some embodiments, the sample passageway is in fluid communication with the outlet of the biochip and the discharge passageway; wherein the sample passageway is positioned between the outlet of the biochip and the discharge passageway.

In some embodiments, the biochip includes an adhesive positioned within the trapping chamber.

In some embodiments, the biochip includes a photonic biogel positioned within the trapping chamber, wherein the photonic biogel comprises a plurality of nanoprobes distributed within a biogel.

In some embodiments, the plurality of nanoprobes comprise gold nanoparticles, and wherein the gold nanoparticles are functionalized with a capture moiety.

In some embodiments, the biochip is removable from the detection device and replaceable with a second biochip.

In some embodiments, the system further includes a user device including a display, wherein the controller is in electronic communication with the user device.

In some embodiments, the biochip is one of a plurality of biochips, wherein the system further includes an air diverter positioned in fluid communication with the supply passageway and the plurality of biochips.

One aspect of the present disclosure provides a method of detecting an airborne particle. The method comprising the steps of: supplying an airflow to a device to generate a negative pressure; collecting an air sample with the negative pressure; capturing airborne particles in the air sample within a biochip positioned within the device; measuring an optical transmission value of the biochip; and analyzing the optical transmission value to detect the airborne particle.

In some embodiments, the method further includes displaying the detection of the airborne particle on a user device.

In some embodiments, the optical transmission value is a change in optical transmission under light illumination from a light source.

In some embodiments, the biochip is a first biochip, and wherein the method further comprises removing the first biochip from the device and inserting a second biochip into the device.

One aspect of the present disclosure provides a photonic biogel for spectroscopic detection of an airborne pathogen. The photonic biogel comprising: a biogel comprising a cross-linked material; and a plurality of plasmonic nanoprobes distributed within the biogel. The plurality of plasmonic nanoprobes are functionalized with a capture moiety that binds to an airborne pathogen.

In some embodiments, the plurality of plasmonic nanoprobes are distributed substantially uniformly throughout the biogel.

In some embodiments, the cross-linked material comprises a gel precursor and a cross-linking agent, wherein the ratio of the gel precursor to the cross-linking material is about 0.5 to about 2.0 (w/w).

In some embodiments, the ratio of the gel precursor to the cross-linking agent is about 0.5 (w/w).

In some embodiments, the plurality of plasmonic nanoprobes have an optical density within the biogel of about 0.05 to about 5.0.

In some embodiments, the plurality of plasmonic nanoprobes have an optical density within the biogel of about 2.0.

In some embodiments, the ratio of the gel precursor to the cross-linking agent is about 0.5 (w/w) and wherein the plurality of plasmonic nanoprobes have an optical density within the biogel of about 2.0.

In some embodiments, the airborne pathogen is a virus.

In some embodiments, the virus is SARS-CoV-2.

In some embodiments, the capture moiety is an antibody.

In some embodiments, the photonic biogel is for use in a method of spectroscopically detecting an airborne pathogen.

In some embodiments, the method of spectroscopically detecting an airborne pathogen comprises the steps of: obtaining a baseline optical transmission value of the photonic biogel; exposing the photonic biogel to an environment having or suspected of having the airborne pathogen; and obtaining a second optical transmission value of the photonic biogel following exposure to the environment, wherein a decrease in the second optical transmission value compared to the baseline optical transmission value indicates that the airborne pathogen is present in the environment.

One aspect of the present disclosure provides a method of spectroscopically detecting an airborne pathogen, the method comprising the steps of: providing a photonic biogel, wherein the photonic biogel comprises a biogel comprising cross-linked material and a plurality of plasmonic nanoprobes distributed within the biogel, wherein the plurality of plasmonic nanoprobes are functionalized with a capture moiety that binds to the airborne pathogen; obtaining a baseline optical transmission value of the photonic biogel; exposing the photonic biogel to an environment having or suspected of having an airborne pathogen; and obtaining a second optical transmission value of the photonic biogel following exposure to the environment, wherein a decrease in the second optical transmission value compared to the baseline optical transmission value indicates that the airborne pathogen is present in the environment.

In some embodiments, the plurality of plasmonic nanoprobes are distributed substantially uniformly throughout the biogel.

In some embodiments, the cross-linked material comprises a gel precursor and a cross-linking agent, wherein the ratio of the gel precursor to the cross-linking material is about 0.5 to about 2.0 (w/w).

In some embodiments, the ratio of the gel precursor to the cross-linking agent is about 0.5 (w/w).

In some embodiments, the plurality of plasmonic nanoprobes have an optical density within the biogel of about 0.05 to about 5.0.

In some embodiments, the plurality of plasmonic nanoprobes have an optical density within the biogel of about 2.0.

In some embodiments, the ratio of the gel precursor to the cross-linking agent is about 0.5 (w/w) and wherein the plurality of plasmonic nanoprobes have an optical density within the biogel of about 2.0.

In some embodiments, the airborne pathogen is a virus.

In some embodiments, the capture moiety comprises an antibody.

In some embodiments, the virus is SARS-CoV-2.

In some embodiments, the airborne pathogen is a gram-negative bacteria.

In some embodiments, the capture moiety comprises a cysteine molecule.

Other aspects of the disclosure will become apparent by consideration of the detailed description and accompanying drawings.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying figures and examples are provided by way of illustration and not by way of limitation. The foregoing aspects and other features of the disclosure are explained in the following description, taken in connection with the accompanying example figures (also “FIG.”) relating to one or more embodiments.

FIGS. 1A-B illustrate a system for integrated airborne particle sample collection and detection for micro-climate quality monitoring.

FIG. 1A illustrates microclimate climate quality monitoring using the integrated airborne particle collection and detection device. The quality is monitored using a smartphone-based wireless display and communication.

FIG. 1B is a schematic diagram that shows an integrated system of air sample collection and a miniaturized detection to assess the level of the airborne particle inside a shared microclimate space. The entire platform is integrated into the airborne particle collection device with a biosensor device that consists of a micro biochip, an optoelectronic detection architecture, and a wireless communication module (e.g., Bluetooth and WiFi) transmitting the acquired data to a smartphone. The micro biochip inducing shear stress traps airborne particles into a polymer layer based on the pressure sensitive adhesive chemistry in the chamber, accordingly.

FIGS. 2A-E illustrate an airborne particle collection device.

FIG. 2A are schematics of the airborne-pathogen collection based on Venturi effect. The airborne particle collection device includes an air supply passageway (e.g., an air supply inlet), a pressure sensor (e.g., a vacuum gauge), a sample passageway (e.g., a sampling port) and a discharge passageway (e.g., a discharge outlet).

FIG. 2B illustrates the pressure distribution in the airborne particle collection device at D_{c}/Dₐᵢᵣ=0.2 and Pₐᵢᵣ=0.1 MPa.

FIG. 2C illustrates the D_{c}/Dₐᵢᵣ effect on η_{C} at Pₐᵢᵣ=i) 10⁻¹, ii) 10⁻², iii) 10⁻³, and iv) 10⁻⁴ MPa.

FIG. 2D illustrates measured P_{c}.

FIG. 2E illustrates ΔP_{c} as a function of Pₐᵢᵣ and d_{particle}, respectively.

FIGS. 3A-D illustrate an airborne particle detection biochip.

FIG. 3A illustrates the detection biochip (scale bar=2 mm).

FIG. 3B illustrates a schematic illustration of the airborne particle trapping in the biochip. i) In the chamber expanded suddenly, the inertia force driven drag force leads to move the airborne particles forward to the bottom of the chamber. ii) a pressure-sensitive polymer (e.g., butyl acetate) is placed on the bottom of the chamber. iii) The drag force of the airborne particle on the pressure-sensitive adhesive results in polymerization of the adhesive layer and strong bonding. iv) The strong binding between the adhesive layer and the airborne particles allows avoiding the detachment of the trapped the airborne particles, although local vortex or swirling of the airflow can occur.

FIG. 3C is visualization of velocity distribution (color profile), particle trajectory (white curves), and streamline (grey curves) at Re=5 and 500.

FIG. 3D is a visualization of velocity distribution (color profile), particle trajectory (white curves), and streamline (grey curves) at various geometry of L/d=10 and 20 at h₀ (=0.5 mm)/d=1 and L/d=0.5 and 10 at h₀(=0.5 mm)/d=5.

FIG. 3E is a graph of Particle trapping efficiency estimated without consideration of the adhesion layer on the bottom of the chamber as a function of L/d from 1 to 10 at h₀=0.5 mm and variation of Re between 50 and 5,000.

FIG. 3F are photo images of trapped microparticles in the biochip (h₀=0.5 mm, d=4 mm, and L=12 mm) at d_{particle}=10⁶ particles and Re=i) 0, ii) 5, and iii) 50 without adhesive layer and Re=iv) 0, v) 5, and vi) 50 with an adhesive layer (Scale bar=2 mm).

FIGS. 4A-C illustrate an integrated microparticle detection system.

FIG. 4A is a photograph image of the integrated detection system. The microparticle contained air sample is loaded into the biochip inlet via negative pressure induced by the airborne particle collection device connected at the outlet of the biochip.

FIG. 4B is a cross-sectional view illustrating alignment between an upper light source, a biochip trapping airborne particles, and a underneath CMOS detector.

FIG. 4C illustrates operation of i) Enclosed detection system and biochip, ii) Detection loaded biochip, iii) wireless communication between the integrated detection system and a smartphone, and iv) an application-software (Bylnk Internet on thing (IoT) Platform) for real-time data acquisition and display.

FIGS. 5A-F illustrate airborne pathogen particle detection tests.

FIG. 5A is a SEM image of trapped Escherichia coli (E. coli) particles in the biochip (h₀=1 mm, d=4 mm, and L=12 mm) at Vᵢₙₗₑₜ=0.01 m/sec (scale bar=1 μm) and ii) photograph images of the surface of the same dimension of biochips as a function of d_{E. coli} from 0 to 10⁸ CFU/mL.

FIG. 5B is a graph of dynamic ΔI/I₀ as a function of d_{E. coli} at i) 10³ ii) 10⁴, iii) 10⁵, iv) 10⁶, v) 10⁷, and vi) 10⁸ CFU/mL.

FIG. 5C is a calibration curve of E. coli detection in the integrated detection system.

FIG. 5D is a calibration curve of Bacillus subtilis detection in the integrated detection system.

FIG. 5E is a calibration curve of Micrococcus luteus detection in the integrated detection system.

FIG. 5F is a calibration curve of Staphylococcus detection in the integrated detection system.

FIGS. 6A-B illustrate validation of nanoprobe detection capability.

FIG. 6A is a graph of localized surface plasmon resonance spectra of the plasmonic nanoprobe (black curve) and the mixture of plasmonic nanoprobe and S-protein (C_{S-protein}=10 ng/mL) (red curve).

FIG. 6B is a calibration curve for S-protein detection of using the integrated optoelectronic biosensor unit at λ=650 nm and P=0.5 mW.

FIGS. 7A-B are a characterization of photonic-biogel samples prepared by a co-polymerization.

FIG. 7A are photograph images of prepared photonic-biogel samples (scale bar=500 μm).

FIG. 7B are graphs of UV-Vis spectra of the photonic-biogel samples as a function of R_{m-c} of the biogel and OD of the plasmonic nanoprobes.

FIGS. 8A-B are a characterization of the photonic-biogel samples prepared by a b-staged co-polymerization.

FIG. 8A are photograph images of prepared photonic-biogel samples (scale bar=500 μm).

FIG. 8B are graphs of UV-Vis spectra of the photonic-biogel samples as a function of R_{m-c} of the biogel and OD of the plasmonic nanoprobes.

FIGS. 9A-B illustrate validation of the photonic-biogel integrated detection system.

FIG. 9A are graphs of measured photoresponse of the photonic-biogel samples as a function of OD of the plasmonic nanoprobe in the photonic-biogel under light off/on/off condition.

FIG. 9B illustrates quantified photocurrent variation as a function of OD of the plasmonic nanoprobe in the photonic-biogel.

FIGS. 10A-B illustrate a microfluidic biochip with integration of a photonic-biogel.

FIG. 10A illustrates the microfluidic biochip design with a cylindrical shaped chamber having a diameter, d and a height, h.

FIG. 10B illustrates the effect of the geometry change on the detection sensitivity; calibration curves for methylene blue as a function of the chamber height from 2 and 4 mm.

FIGS. 11A-C illustrate detection performance of SARS-CoV-2 particles in the photonic-biogel.

FIG. 11A is a graph of dynamic binding curves of SARS-CoV-2 particles at different concentrations from 0.001 to 10 pfu/μL,

FIG. 11B is a graph of a calibration curve for SARS-CoV-2 in the photonic-biogel.

FIG. 11C is a bar chart of the biosensor signal for PBS solutions spiked with SARS-CoV-2 particles and control particles (SiO₂ nanoparticle) at various concentrations.

FIGS. 12A-B illustrate detection performance of E. coli particles.

FIG. 12A are SEM images of E. coli samples with control particle (conventional AuNPs) and synthesized plasmonic nanoprobes.

FIG. 12B is a graph of Measured OD for the mixture of the plasmonic nanoprobes and E. coli as a function of E. coli density from 10⁴ to 10⁸ cfu/mL.

FIG. 13A-B is a schematic illustration of an integrated airborne particle sample collection and detection for micro-climate quality monitoring. (a) Micro-climate quality monitoring using the integrated airborne particle collection and detection. The quality is monitored using a smartphone-based wireless display and communication. (b) The schematic shows an integrated system of air sample collection and a miniaturized detection of the population of airborne particles inside a shared microclimate space. The entire platform integrates the airborne particle collection device with a biosensor device that consists of a micro biochip, an optoelectronic detection architecture, and a wireless communication module (e.g., Bluetooth) transmitting the acquired data to a smartphone. The micro biochip induced shear stress traps airborne particles into a polymer layer based on the pressure sensitive adhesive chemistry in the chamber, accordingly.

FIG. 14A-C illustrates a design of an integrated air quality monitoring system. (a) The entire system integrates an air sample collector with a detection unit that consists of three groups of light sources, photodetectors, and a microcontroller embedded with a Bluetooth communication module. The microcontroller receives commands from an app on the user's smartphone to start or stop the detection process and sends real-time detection results to the smartphone on which they are shown as concurrently updating plots. The biochips in each channel contain antibodies for the target virus. (b) The integrated air hander (air sample collector) uses certain amount of air supply to generate a negative pressure that drags the surrounding airflow outside, into the detection device. The schematic shows a simulation of airflow inside the collector. (c) The integrated air quality monitoring system is aimed to detect potential aerosols containing SARS-CoV-2 in an enclosed space such as a vehicle. The air quality is monitored using a smartphone-based Bluetooth communication.

FIG. 15 illustrates an integrated detection system including a detection unit, an air handler, an inlet, and an outlet. The biochips are loaded into the manual sample loader. The detection unit contains three channels of detection parts. Individual detection parts are assembled with two microfluidic connectors, an LED, a photodetector, a biochip, an optofluidic aligner, and a photodetector.

FIG. 16A-B illustrates application software for smartphone-based data collection and display. (a) Circuit block diagram of the integrated system. (b) Version 1 is based on Blynk platform; version 2 application software have been upgraded for android based operation.

FIG. 17A-B illustrates a system level pressure test using the integrated system. The integrated air handler enables the generation of negative pressure accurately as a function of inlet flow.

FIG. 18A-B is a system level optical signal ON/OFF test using the integrated system. The photodetector effectively detected the change in light intensity between light ON/OFF states in the integrated system.

FIG. 19 is a graph illustrating confirmation of signal stability. The influence on light absorbance change with control cases including photonic-biogel with SiO₂ NP as a function of the SiO₂ NP population.

FIG. 20A-B illustrates system level detection performance of virus particles in the integrated system. (a) Calibration curve for virus in the integrated systems, (b) Dynamic binding curves of SARS-CoV-2 at different populations achieved by the integrated system.

FIG. 21 is a graph of system level detection performance of bacteria particles in the integrated system.

Before any embodiments are explained in detail, it is to be understood that the invention is not limited in its application to the details of construction and the arrangement of components set forth in the following description or illustrated in the following drawings. The invention is capable of other embodiments and of being practiced or of being carried out in various ways.

### DETAILED DESCRIPTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e., at least one) of the grammatical object of the article. By way of example, “an element” means at least one element and can include more than one element.

“About” and “approximately” are used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that an apparatus comprises components A, B, and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

The rapid emergence of air-mediated diseases in micro-climates demands on-site monitoring of airborne particles. Such detection of airborne particles becomes more necessitating as the particles are highly localized and dynamically change over time. However, conventional monitoring systems rely on time-consuming sample collection and centralized off-site analysis.

The disclosure herein provides a smartphone based integrated system (microsystem) for on-site collection and detection that enables real-time detection of indoor airborne particles with high sensitivity. A Venturi-based collection device is designed to collect airborne particles without requiring an additional power supply. The present disclosure provides the collection device to collect airborne particle dispersed in the microclimate air.

Systematic analysis shows that the collection device collects microparticles with consistent negative pressure, regardless of the particle concentration in the air sample. By incorporating a microfluidic-biochip based on inertial force to trap particles and an optoelectronic photodetector into a miniaturized device with a smartphone, real-time and sensitive detection is achieved of the collected airborne particles, such as Escherichia coli, Bacillus subtilis, Micrococcus luteus, and Staphylococcus with a particle-density dynamic range of 10³-10⁸ CFU/mL. Because the system disclose herein is capable of minimal-power sample collection, high sensitivity, and rapid detection of airborne particles, the system can be readily adopted by the government and industrial sectors to monitor indoor air contamination and improve human healthcare.

Stabilized and consistent operation of the collection device is demonstrated herein with a broad range of the airborne particles density in airflow. Furthermore, the entire platform integrates the collection device with an optoelectronic detection device that consists of a microfluidic particle trapping chamber and a complementary metal-oxide-semiconductor (CMOS) photodetector under a smartphone based communication. The platform effectively collects and detects airborne particle such as Escherichia coli (E. coli), Bacillus subtilis, Micrococcus luteus, and Staphylococcus of varying particle densities (from 10³ to 10⁸ CFL/mL) in less than one minute.

Microfluidic-device-based detection methods reduce time, sample volume, and labor requirements typical of the conventional approaches. However, conventional microfluidic devices manifest sample-sparing and high-sensitivity detection capabilities, but their operations still require a liquid-phase sample, large-scale equipment, or many auxiliary parts to provide the functions of sample collection, reagent treatment steps, and data processing. As such, conventional approaches prohibit the direct collection and detection of airborne parties in a gas phase.

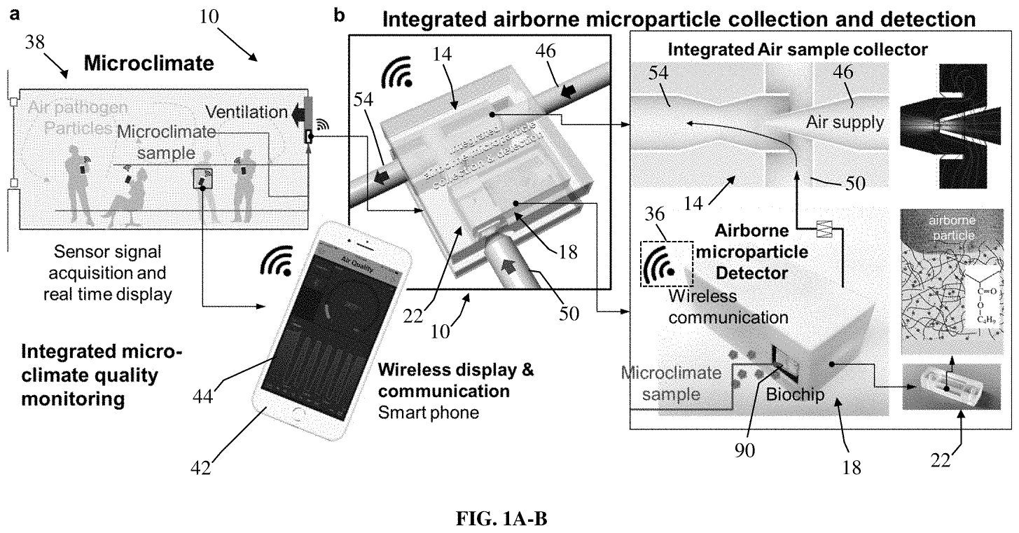

With reference to FIG. 1, a system 10 for on-site collection and detection of airborne particles for microclimate quality control is illustrated. The system 10 is a micro-climate quality monitoring system. The air monitoring system 10 includes a venturi pump 14 (i.e., a collection device) and a detection device 18 including a biochip 22, a light emitting source 26, a photodetector 30 and a controller 34 electronically coupled to the photodetector 30. The system 10 is a smartphone-based integrated airborne particle collection and detection system with automatic, real-time, and sensitive analysis.

FIG. 1a illustrates the system 10 is positioned in a microclimate 38 for microclimate climate quality monitoring using the integrated airborne particle collection device 14 and detection device 18. In some embodiments, the quality is monitored using a smartphone 42 and smartphone-based wireless display and communication.

FIG. 1b illustrates a schematic diagram of the integrated system 10 of air sample collection and a miniaturized detection to access the level of the airborne particle inside a shared microclimate space (e.g., microclimate 38). The platform is integrated into the airborne particle collection device with a biosensor device that consists of the micro biochip, the optoelectronic detection architecture, and the wireless communication module (e.g., Bluetooth) transmitting the acquired data to a smartphone. As explained in further detail below, the micro biochip inducing shear stress traps airborne particles into a polymer layer based on the pressure sensitive adhesive chemistry in the chamber.

### Airborne Particle Collection Device

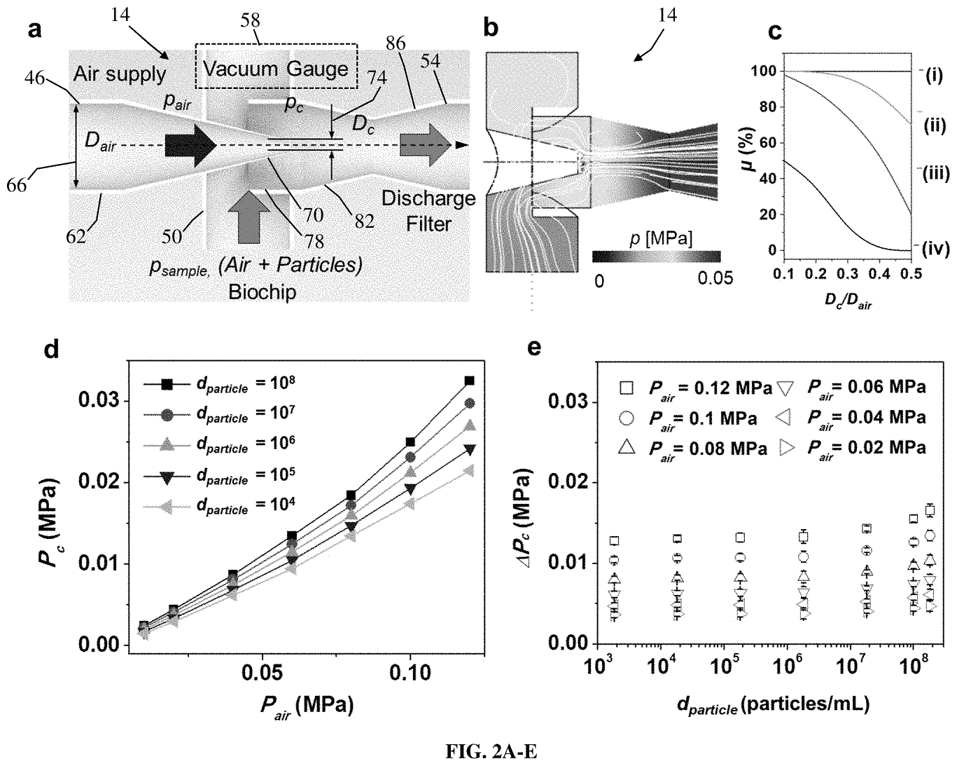

With reference to FIG. 2, the integrated airborne particle collection device in the illustrated embodiment is the venturi pump 14. The venturi pump 14 includes an air supply passageway 46, a sample passageway 50, and a discharge passageway 54. In the illustrated embodiment, the discharge passageway 54 is in fluid communication with the air supply passageway 46 and the sample passageway 50. In some embodiments, the sample passageway 50 is oriented at an angle with respect to the air supply passageway 46. In the illustrated embodiment, the sample passageway 50 is orthogonal to the air supply passageway 46. In some embodiments, the system 10 further includes a pressure sensor (e.g., a pressure gauge 58) coupled to the sample passageway 50.

The air supply passageway 46 includes a first portion 62 with a first diameter 66 and a second portion 70 with a second diameter 74 that is smaller than the first diameter 66. In some embodiments, a ratio of the second diameter 74 to the first diameter 66 is approximately 0.2. In the illustrated embodiment, the second portion 70 is positioned within the discharge passageway 54. The discharge passageway 54 includes a cylindrical portion 78, a first tapered portion 82, and a second tapered portion 86. In the illustrated embodiment, the first tapered portion 82 (portion decreasing in diameter in the direction of airflow) is positioned between the cylindrical portion 78 and the second tapered portion 86 (portion increasing in diameter in the direction of airflow).

FIG. 2a are schematics of the airborne-pathogen collection based on Venturi effect. The airborne particle collection device 14 includes the air supply passageway 46 (e.g., an air supply inlet, the pressure sensor (e.g., the vacuum gauge 58), the sample passageway 50 (e.g., a sampling port) and a discharge passageway 54 (e.g., a discharge outlet).

FIG. 2b illustrates the pressure distribution in the airborne particle collection device at D_{c}/Dₐᵢᵣ=0.2 and Pₐᵢᵣ=0.1 MPa.

FIG. 2c illustrates the D_{c}/Dₐᵢᵣ effect on η_{C} at Pₐᵢᵣ=i) 10⁻¹, ii) 10⁻², iii) 10⁻³, and iv) 10⁻⁴ MPa.

FIG. 2d illustrates measured P_{c}.

FIG. 2e illustrates ΔP_{c} as a function of Pₐᵢᵣ and d_{particle}, respectively.

The airborne particle collection device disclosed herein (e.g., the venturi pump) advantageously does not need additional pumps. Conventional pumping steps for the collection of air samples hinders miniaturization and power effectiveness. As disclosed herein, the airborne particle collection device is intended to operate by means of the Venturi effect in a miniaturized platform to enable on-site sampling of airborne particle. Since most indoor and microclimates involve heating, ventilation, and air conditioning (HVAC) system, by utilizing air flow generated by such HVAC systems in the indoor microclimate, the airborne particles are collected without additional power input to generate air flow. In some embodiments, a building HVAC system is fluidly coupled to the air supply passageway 46.

When air is supplied to the collection device 14 through the inlet, a rapid pressure drop occurs around the inner constricted section of the device channel. The rapid pressure drop induces a suction of air containing airborne particles from the micro-climate environment. The airflow from the supply and sample sides are discharged through the outlet thereafter. The theoretical pressure drop due to Venturi effect at the constriction is estimated by Pₐᵢᵣ−P_{c}=ρₐᵢᵣ/2 (v_{c}²−vₐᵢᵣ²), where Pₐᵢᵣ is the pressure at the air supply inlet, P_{c} is the pressure of the constricted section, Pₐᵢᵣ is the density of air, v_{c} is the velocity of the constricted section, and vₐᵢᵣ is the velocity of the air flow. Here, Pₐᵢᵣ and the ratio between the diameter of the air supply inlet (Dₐᵢᵣ) and that of the constricted section (D_{c}), were utilized as design parameters. To set up the airborne particle collection device in a micro-climate, the inside of the motor vehicle was targeted utilized to define the overall scale of the airborne particle collection device 14 (e.g., Width×Height×Depth<4×2×2 cm³).

The concept of the airborne particle collection device 14 using FEA is confirmed (FIG. 2b). In this analysis, the particle trajectories are calculated by solving the ballistic equation of motion for each microparticle. It was assumed that a spherical particle with a mass (m) and a radius (rₚ) in the air flow with a density (ρ) and a dynamic viscosity (η) was subjected to gravitational forces, the hydrodynamic Stokes' drag, and the related buoyancy. The fluid drag force was estimated from the Khan and Richardson force, which is given for a wide range of Reynolds numbers (Re) as:

F=πrₚ²ρ(ū−ūₚ)²(1.84Reₚ^{-0.31}+0.293Reₚ^{0.06})^{3.45} (EQN. 1)

Reₚ=(|ū−ūₚ|2rₚρ)/η (EQN. 2)

where, u represents the fluid velocity, and uₚ represents the particle velocity. To predict the fluid drag force based on EQN. 1, the incompressible Navier-Stokes equation and the continuity equation are solved to obtain the air velocity field, uₚ and P in the airborne particle collection device. Considering the asymmetric shape of the device, the computational system was constructed using a 3D model. The pressure distribution and particle tracing at D_{c}/Dₐᵢᵣ=0.2 (Dₐᵢᵣ=1 cm), Pₐᵢᵣ=10⁻² MPa shows that P_{c} generation results in the induction of particles (rₚ=1 μm) flowing from the micro-climate side. The particle collection efficiency is also estimated (η_{C}=n_{c}/nₛ×100%, where ns is the number of particles injected into the sampling area and n_{c} is the number of particles at the constriction area), as a function of D_{c}/Dₐᵢᵣ and Pₐᵢᵣ (FIG. 2c). The estimated η_{C} rapidly increases from 0 to 100% as either Pₐᵢᵣ increases from 10⁻⁴ to 10⁻¹ at D_{c}/Dₐᵢᵣ is 0.1 or D_{c}/Dₐᵢᵣ decreases from 0.5 to 0.1 when Pₐᵢᵣ is above 10⁻² MPa. FEA results indicate that Venturi effect in the airborne particle collection device design allows high negative pressure to induce particle collection in airflow for the miniaturized platform.

To confirm the airborne particle collection device 14 operation at the different densities of airborne particles in the microclimate, the negative pressure at the constricted zone was first measured by installing the vacuum gauge 58 in the airborne particle collection device when air is injected into the air supply inlet. As a representative microparticle, 1 μm-dia. polystyrene (PS) beads were tested along with varying densities (d_{particle}). FIG. 2d plots the measured P_{c} as a function of Pₐᵢᵣ from 0 to 0.12 MPa at varying d_{particle} from 104 to 10 particles/mL. P_{c} increased with Pₐᵢᵣ. In addition, higher d_{particle} led to higher P_{c}, however variation of P_{c} (ΔP_{c}=P_{c_air}−P_{c_particle}, where P_{c_air} and P_{c_particle} are the pressure of constricted section of airflow without and with particles, respectively) as a function of d_{particle}, wherein between 10³ and 10⁸ particles/mL/does not show significant changes (FIG. 2e). For instance, even at Pₐᵢᵣ=0.08 MPa, ΔP_{c} varied from 0.007 to 0.01 MPa with d_{particle} ranging from 10³ to 10⁸ particles/mL. In such a wide range of d_{particle}, the airborne particle collection device enables the collection of airborne particles without any clogging and dramatic change of pressure issues.

### Biochip

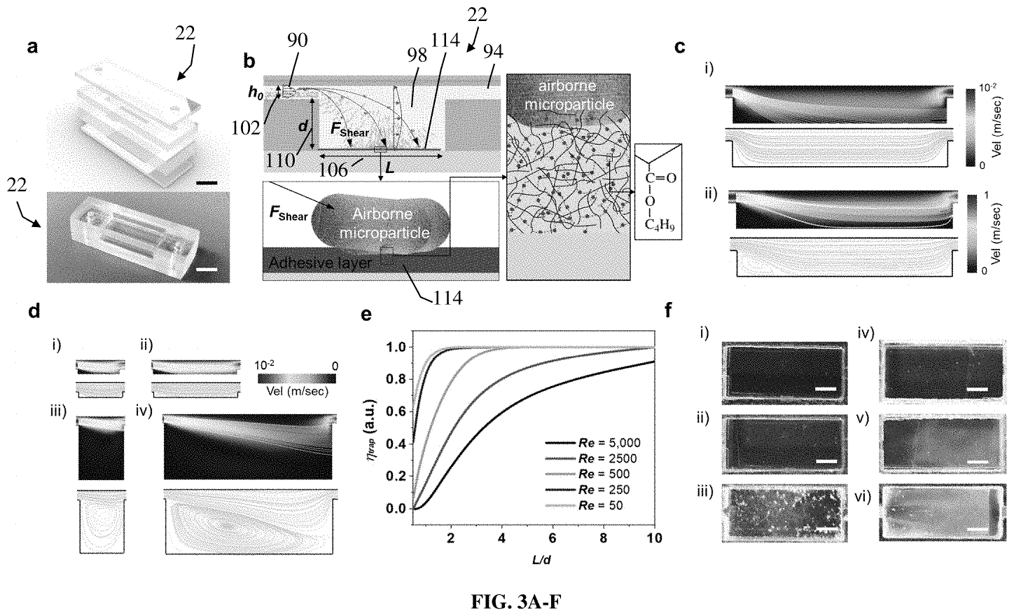

With reference to FIG. 3, the airborne particle detection biochip 22 includes an inlet 90, an outlet 94, and a trapping chamber 98 positioned between the inlet 90 and the outlet 94. In the illustrated embodiment of FIG. 3, the inlet 90 defines a height 102 and a trapping chamber 98 is rectangular and includes a length 106, and a depth 110. In some embodiments, a ratio of the length 106 to the depth 110 is within a range of approximately 1 to approximately 10. In the illustrated embodiment, the sample passageway 50 is in fluid communication with the outlet 94 of the biochip 22 and with the discharge passageway 54. In the illustrated embodiment, the sample passageway 50 is positioned between the outlet 94 of the biochip 22 and the discharge passageway 54. In other words, the sample passageway 50 is positioned downstream of the outlet 94 of the biochip 22. As discussed further herein, in some embodiments, the biochip 22 includes an adhesive 114 positioned within the trapping chamber 98.

FIG. 3a illustrates the detection biochip 22 (scale bar=2 mm).

FIG. 3b illustrates a schematic illustration of the airborne particle trapping in the biochip. i) As the chamber expands suddenly, the inertia force driven drag force leads to move the airborne particles forward to the bottom of the chamber. ii) a pressure-sensitive polymer (e.g., butyl acetate) is placed on the bottom of the chamber. iii) The drag force of the airborne particle on the pressure-sensitive adhesive results in polymerization of the adhesive layer and strong bonding. iv) The strong binding between the adhesive layer and the airborne particles allows avoiding the detachment of the trapped the airborne particles, although local vortex or swirling of the airflow can occur.

FIG. 3c is visualization of velocity distribution (color profile), particle trajectory (white curves), and streamline (grey curves) at Re=5 and 500.

FIG. 3d is a visualization of velocity distribution (color profile), particle trajectory (white curves), and streamline (grey curves) at various geometry of L/d=10 and 20 at h₀ (=0.5 mm)/d=1 and L/d=0.5 and 10 at h₀(=0.5 mm)/d=5.

FIG. 3e is a graph of Particle trapping efficiency estimated without consideration of the adhesion layer on the bottom of the chamber as a function of L/d from 1 to 10 at h₀=0.5 mm and variation of Re between 50 and 5,000.

FIG. 3f are photo images of trapped microparticles in the biochip (h₀=0.5 mm, d=4 mm, and L=12 mm) at d_{particle}=10⁶ particles and Re=i) 0, ii) 5, and iii) 50 without adhesive layer and Re=iv) 0, v) 5, and vi) 50 with an adhesive layer (Scale bar=2 mm).

The integrated airborne particle detection system 10 demonstrates real-time monitoring of the particle density (FIG. 3). The microfluidic biochip includes the inlet, trapping chamber and outlet to capture airborne particles in the air sample using a concept of the inertia flow (FIG. 3a). The inertia flow concept enables the operation of the detection system with minimal pressure drop, unlike conventional systems based on the filtering. The minimal pressure drop, also does not need pumping and mechanical parts to support high pressure operation, allowing for a miniaturized detection system. Conventional inertial flow based microfluidic device focus on aqueous phases and larger microparticles (rₚ>5 μm). Low viscosity airflow leads to eddy flows or vortexes in the micro device, creating challenges to trapping airborne particles in the microchip.

In the microfluidic biochip 22, the collected air sample enters the inlet 90 (e.g., a narrow inlet channel) and then flows into the trapping chamber 98 (e.g., a larger chamber space). In the larger height chamber, the velocity of the particles becomes slower due to a larger gravitational force and lower kinetic energy. This leads to the settling of the particles at the bottom of the microfluidic chamber 98. The pressure-sensitive polymer 114 (butyl acetate) is placed on the bottom of the chamber. The shear stress between the airborne particle and the pressure-sensitive adhesive layer 114 results in polymerization of the adhesive layer and strong bonding. This strong binding allows avoiding the detachment of the trapped airborne particles, although local vortex or swirling of the airflow (FIG. 3b).

The velocity profiles, particle trajectories, and streamlines for different and the particle-capture efficiency in the designed biochip are estimated for different inlet velocities and chamber geometries using FEA (FIGS. 3c and 3d). Lower inlet velocity (Re=50) results in a symmetric velocity profile and settling the particles on the bottom of the chamber, while a higher one (Re=500) shows an asymmetric velocity profile with fewer numbers of settling particles. The geometric effect of channel height (h₀)=1 mm, chamber depth (d)=4 mm, and chamber length (L) on the particle trapping into the chamber is also considered. Larger eddy occurs with d/h₀, while L/d does not show clear trends in the eddy size. Meanwhile, longer L/d leads to a larger number of particle trapping in the micro chamber 98. Based on the analysis results, particle capturing efficiency (ηₜᵣₐₚ) is close to 100% when Re at the inlet is kept below 250 at d=4 mm, and chamber length L=12 mm when h₀ is 1 mm (FIG. 3e). Furthermore, the high ratio of the channel length to depth (L/d>3) leads to ηₜᵣₐₚ=approximately 100%. Once the flowing particles touch the bottom-/side-wall of the biochip chamber, they are affected by shear stress. This means that they are captured effectively. Furthermore, to ensure the remaining attachment of the captured airborne particles, the pressure sensitive adhesion layer 114 is positioned on the bottom surface of the microfluidic chamber (FIG. 3f).

Minimal aggregation in the biochip 22 is achieved with the adhesive layer 114, meanwhile, the particle aggregation existed when there was no adhesion layer (control). The adhesive layer 114 in the biochip led to uniform distribution and less particle aggregation. The total number of airborne particles retained in the chamber can be determined by the loading time and the concentration of microparticle in suspension. This means that the biochip 22 is an indicator of the number of the microparticle in the airflow.

### Integrated Airborne Particle Detection Device

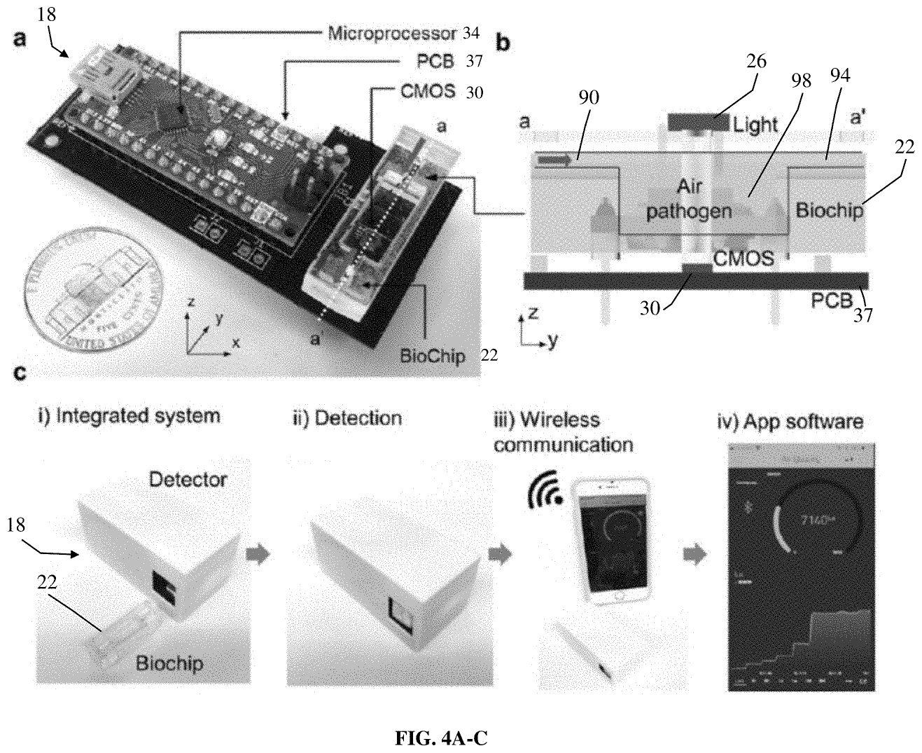

With reference to FIG. 4, the detection device 18 includes the biochip 22, the light emitting source 26 (e.g., a light emitting diode LED, a laser, etc.), the photodetector 30 (e.g., a CMOS photodetector), the controller 34 (e.g., a microcontroller unit MCU), a communication module 36 (e.g., a Bluetooth® module), and a printed circuit board 37 (PCB). In some embodiments, the microfluidic biochip 22 is removable from the detection device 18 and replaceable with a second microfluidic biochip. In some embodiments, the user device 42 (e.g., a cell phone, a laptop, a tablet, etc.) includes a display 44 and the controller 34 is in electronic communication (e.g., wired or wireless) with the user device 42 by the communication module 36.

FIG. 4 illustrates an integrated microparticle detection system and device.

FIG. 4a is a photograph image of the integrated detection device 18. The microparticle contained air sample is loaded into the biochip inlet 90 via negative pressure induced by the airborne particle collection device 14 connected at the outlet 94 of the biochip 22.

FIG. 4b is a cross-sectional view illustrating alignment between the upper light source 26, the biochip 22 trapping airborne particles, and the underneath CMOS detector 30.

FIG. 4c illustrates operation of i) Enclosed detection system and biochip, ii) Detection loaded biochip, iii) wireless communication between the integrated detection system and a smartphone, and iv) an application-software (e.g., Internet on thing (IoT) Platform) for real-time data acquisition and display.

The microparticle trapping in the biochip 22 results in changes in the optical transmission that is detected by the CMOS photodetector 30 positioned under light illumination from the top LED or laser 26 (FIG. 4a). The change of optical transmission results in the photocurrent (I) to change through the integrated CMOS photodetector 30. The measured I represents the density of the trapped particles from the airflow collected by the airborne particle collection device 14.

In the illustrated embodiment, operation includes i) supplying air into the airborne particle collection device to generate negative pressure and induce the collection of air sample containing airborne particles from the microclimate, ii) flowing the collected air sample into the biochip, iii) capturing microparticles in the air sample into the biochip, iv) measuring a change of optical transmission under light illumination, v) transmitting the measured optical-signal change to a smartphone, and vi) displaying the measured results through the installed application software (FIG. 4c). After completion of the detection and the data acquisition, the used biochip, in some embodiments, is replaced with a new one for the next measurement. This permits a recycled use of the same CMOS photodetectors and the LED.

The pixel size of the CMOS detector (3 mm×1.5 mm) underneath the micro chamber is large enough to cover more than 50% of the particle detection area (i.e., the bottom surface) of the micro chamber. This allows the detector to measure photo signals spatially averaged over a majority (approximately 80%) of trapped microparticles according to the prediction in FIG. 3c. The detection of the spatially averaged signal is expected to limit the effect of the particle distribution on measurement.

### Operation of Airborne Particle Collection and Detection System

In some embodiments, the present disclosure provides a method of detecting an airborne particle, where the method includes supplying an airflow to a device to generate a negative pressure and collecting an air sample with the negative pressure. The method also includes capturing airborne particles in the air sample within a biochip positioned within the device and measuring an optical transmission value of the biochip. The method also includes analyzing the optical transmission value to detect the airborne particle. In some embodiments, the method further includes displaying the detection of the airborne particle on a user device. In some embodiments, the optical transmission value is a change in optical transmission under light illumination from a light source. In some embodiments, the biochip is a first biochip and the method further includes removing the first biochip from the device and inserting a second biochip into the device.

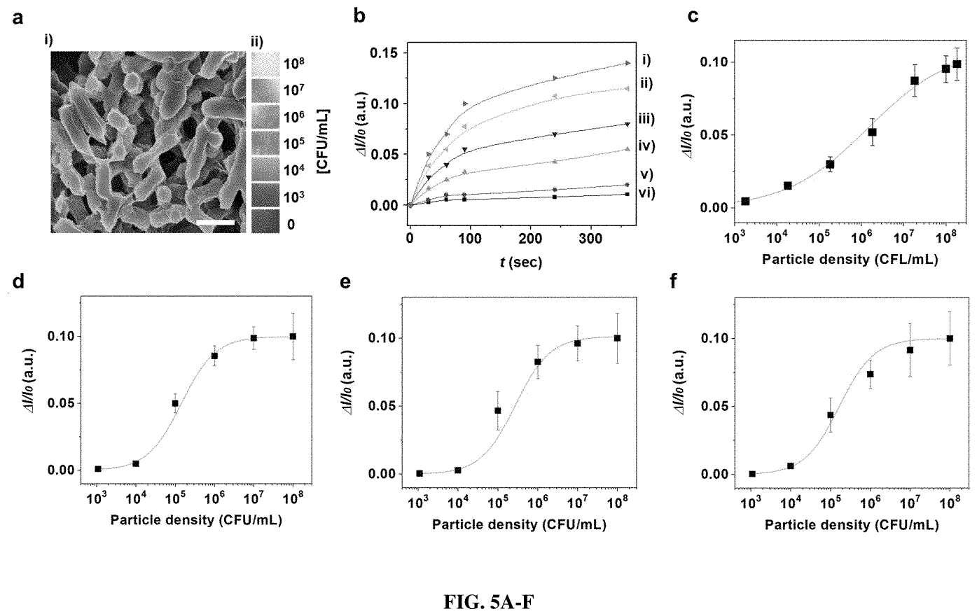

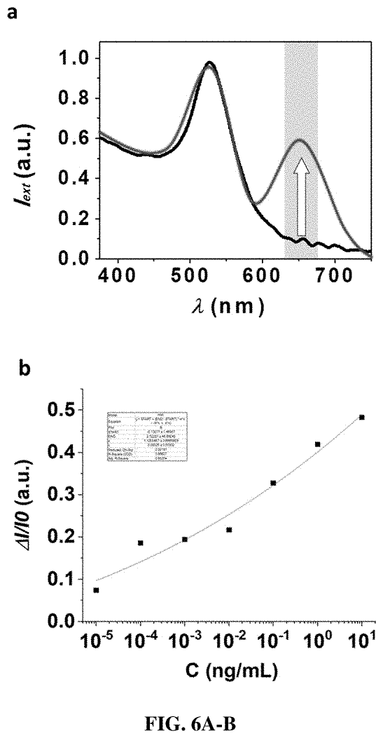

FIG. 5 illustrates airborne pathogen particle detection tests performed.

FIG. 5A is a SEM image of trapped E. coli particles in the biochip (h₀=1 mm, d=4 mm, and L=12 mm) at Vᵢₙₗₑₜ=0.01 m/sec (scale bar=1 μm) and ii) photograph images of the surface of the same dimension of biochips as a function of d_{E. coli} from 0 to 108 CFU/mL.

FIG. 5b is a graph of dynamic ΔI/I₀ as a function of d_{E. coli} at i) 10³′ ii) 10⁴, iii) 10⁵, iv) 10⁶, v) 10⁷, and vi) 10⁸ CFU/mL.

FIG. 5c is a calibration curve of E. coli detection in the integrated detection system.

FIG. 5d is a calibration curve of Bacillus subtilis detection in the integrated detection system.

FIG. 5e is a calibration curve of Micrococcus luteus detection in the integrated detection system.

FIG. 5f is a calibration curve of Staphylococcus detection in the integrated detection system.

During testing, airborne pathogen microparticles of E. coli, Bacillus subtilis, Micrococcus luteus, and Staphylococcus were used as target airborne particles. These airborne pathogens have been clinically known as the critical sources in a variety of infections and severe respiratory diseases. The trapping of the airborne particles in the biochip chamber 98 was validated by obtaining a scanning electron microscope (SEM) image (FIG. 5a (i)). At Pₐᵢᵣ=0.05 MPa, after flowing the airflow sample with 105 CFU/mL of E. coli density (d_{E. coli}) for 60 sec, the obtained SEM image indicates that the experimental setup is configured to collect and trap the airborne particle. The photographic images of the surface of the biochips show that light extinction is varied as a function of d_{E. coli} from 0 to 108 CFU/mL (FIG. 5a (ii)). Then, the airborne particle detection was performed using the integrated system of the airborne particle collection device and the biochip as a function of d_{E. coli}(FIG. 5b). Depending on the airborne particle density, the change of optical transmission in the biochip leads to different photocurrent signals (I) from the CMOS photodetector. The variation of the measured photocurrent (ΔI/I₀) is a function of d_{E. coli}. After loading E. coli samples into the aerogel generator, the air sample flow was generated by the airborne particle collection device, and the ΔI/I₀ was measured as a function of d_{E. coli} from 10³ to 10⁸ CFU/mL. The ΔI/I₀ rapidly increases for the first 60 seconds and gradually reaches a plateau regardless of d_{E. coli}. For instance, when d_{E. coli} is 10⁵ CFU/mL, ΔI/I₀ is 0.025 at t=60 seconds, and slightly increases to 0.05 for next 300 seconds. Quantification of E. coli density in the air samples was enabled in such a short detection time.

Based on the dependency of the ΔI/I₀ on the particle density, a calibration curve of ΔI/I₀ is determined as a function of particle density in the air sample (FIGS. 5c-5f). While the airborne particle collection device operates, the biochip enabled the detection of the airborne pathogens; Bacillus subtilis, Micrococcus luteus, and Staphylococcus from 10³ to 10⁸ CFU/mL. The estimated limit of detection (LOD) of the integrated system of the airborne particle collection device and the biochip for E. coli in an airflow is as low as 411 CFU/mL according to 3σ/kₛₗₒₚₑ (σ is the standard deviation of the background signal measured from a blank control. kₛₗₒₚₑ is the regression slope of the calibration curve). Considering their different shapes, sizes, surface charges, and membrane structures, these airborne pathogens are representative examples only and the disclosure is not limited those pathogens described herein. The estimated LODs of the integrated system for the airborne particles according to the constructed calibration curves are approximately 620, approximately 280, and approximately 400 CFU/mL for Bacillus subtilis, Micrococcus luteus, and Staphylococcus, respectively. These results indicated that the integrated system 10 consistently performed effective collection and detection performance, regardless of the shape, size, and surface charge of the microparticles.

In addition, the sample collector geometry allows particles within a narrow mass range, such as bacteria particles (density=˜1.1166±0.0007 g/ml), to be selectively collected. Specifically, the mass-specific particle collection was arranged by carefully choosing the ratio between the contraction and air supply sizes. To make the integrated sample collection and detection system available for broader use, the effects of the geometry and multistage arrangement of the collector on particle collection can be utilized.

### Integrated COVID-19 Viral and Bacterial Particle Detection for Micro-Climate Quality Monitoring

Coronavirus disease (COVID-19) is an acute respiratory failure-causing airborne disease. Despite the current effort to manage the disease, the rapid spread of the COVID-19 pandemic reflects the fundamental shortcoming in preventing viral infections with existing diagnostic tests. Those diagnostic tests only permit tracing hosts already exposed to viruses existing in the air. The preventative strategy is urgently needed to fight with the COVID-19 pandemic by warning the presence of virus particles in the air prior to their intake by the host.

In another embodiment, a compact and portable biosensor enables direct, rapid, and sensitive detection of airborne virus particles. The device incorporates an air-flow microfluidic biochip 200 with a biofunctional nanoparticle-embedded hydrogel layer called the “photonic-biogel” 204 and a compact micro-optic device. An optical transmission shift of the hydrogel layer upon the binding of airborne virus particles with the nanoparticles is quantitatively correlated with the population of the viruses in the air. The high density and uniform distribution of plasmonic nanoprobes made of biofunctional nanoparticles in the photonic-gel facilitate rapid diffusion of virus particles and strong analyte-nanoparticle interaction, thus yielding the rapid and sensitive response of the device. In addition, systematic design optimization enhances the optical response of the microfluidic device to the transmission change. The operation of this biosensor requires no sample preparation such as purification and dissolution. Detection of airborne viral sample in a large volume of air flow is demonstrated.

An integrated airborne pathogen detector (iAPD) incorporating the micro-optofluidic device 200 integrated with a photonic-biogel 204 layer is illustrated for indoor air quality monitoring. The integrated photonic-biogel is synthesized by copolymerization of the hydrogel and plasmonic nanoprobes formed by biofunctional nanoparticles in the micro-optofluidic device. The airborne particles in the sample are directly quantified by measuring the change in optical transmission originating from the binding between the airborne particle and plasmonic nanoprobes in the photonic-biogel material. To maximize the diffusion of airborne particles and their binding with the plasmonic nanoprobes in the photonic-biogel, the porosity and density of the plasmonic nanoprobes in the structure is optimized. Additionally, to increase the optical response of the transmission change, the geometry microfluidic device is optimized. The iAPD provides direct, rapid, and sensitive detection of airborne particles in the sample without pre-detection steps related to sample purification, liquidation, and lysis involved in current viral and bacterial analysis approaches.

As disclosed herein, the iAPD includes biosensor integrating the photonic-biogel 204 with a micro-optofluidic device. The photonic-biogel 204 includes copolymerization of the hydrogel and plasmonic nanoprobes. The change in optical transmission originating from the binding of the airborne particles (SARS-CoV-2) with plasmonic nanoprobes in the photonic-biogel enabled the quantification of SARS-CoV-2. The porosity and density of the plasmonic nanoprobes are designed to maximize the diffusion of airborne particles and binding with the plasmonic nanoprobes, simultaneously. The height and shape of a microfluidic chamber 208 in the biochip 200 are also optimized to increase the optical response of the transmission change. Using the iAPD, direct, rapid, and sensitive detection of airborne particles (SARS-CoV-2) in the sample is achieved. Simultaneously, bacteria detection is also achieved integrated virus and bacterial particle detection performance in a single detection system.

### S-Protein Detection

FIG. 6 illustrates validation of nanoprobe detection capability.

FIG. 6a is a graph of localized surface plasmon resonance spectra of the plasmonic nanoprobe (black curve) and the mixture of plasmonic nanoprobe and S-protein (C_{S-protein}=10 ng/mL) (red curve).

FIG. 6b is a calibration curve for S-protein detection of using the integrated optoelectronic biosensor unit at λ=650 nm and P=0.5 mW.

The S protein has a well-established role in the assembly of virions where it may induce membrane curvature or aid in membrane scission. As stated above, the S-protein has been recognized as a molecular signature of SARS-CoV-2. An S-protein analysis can indicate the SARS-CoV-2 infection by this process. As the first step of the SARS-CoV-2 particle detection, the design and capability of the prepared plasmonic nanoprobe for the detection of S-protein is confirmed. FIG. 6a shows the spectra of the plasmonic nanoprobe (black curve) and a mixture of the probe and S-protein (C_{S-protein}=10 ng/mL) (red curve). The mixture of the plasmonic nanoprobe and S-protein led to two extinction peaks at approximately 550 and approximately 660 nm, while the probe without S-protein led to a single extinction peak at 532 nm. The second peak at approximately 660 nm is attributed to the nanoprobe-nanoprobe interaction bridged by the S-protein and antibody on the nanoprobes. In addition, FIG. 6b shows a calibration curve as a function of the S-protein concentration in the range of 10⁻⁵ to 10 ng/mL. After incubating the nanoprobe and S-protein solution for 30 minutes, the log-scale plot shows that the photocurrent variation linearly increases with the S-protein concentration; the estimated limit of detection (LOD) is low, 0.4×10⁻⁵ ng/mL. These results indicate that the prepared plasmonic nanoprobes can be used to detect SARS-CoV-2.

### Photonic-Biogel Fabrication

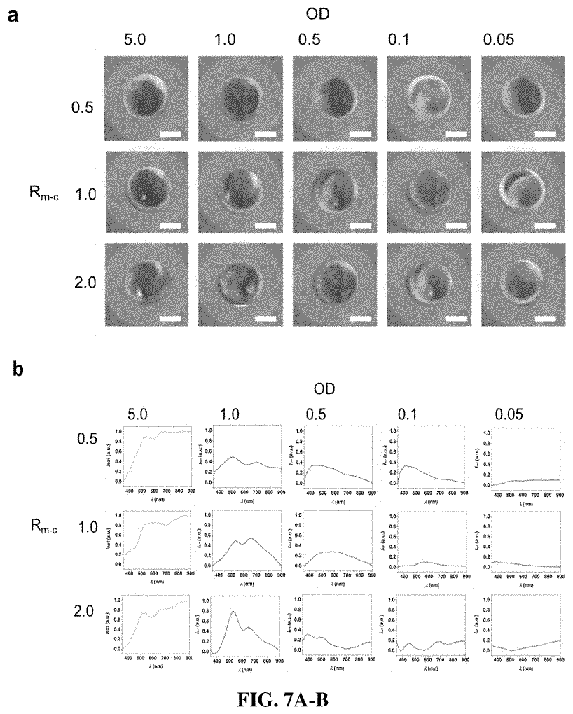

FIG. 7 is a characterization of photonic-biogel samples prepared by a co-polymerization.

FIG. 7a are photograph images of prepared photonic-biogel samples (scale bar=500 μm).

FIG. 7b are graphs of UV-Vis spectra of the photonic-biogel samples as a function of R_{m-c} of the biogel and OD of the plasmonic nanoprobes.

After confirming the detection capability of the plasmonic nanoprobes, the photonic-biogel is formed by incorporating the plasmonic nanoprobes into a hydrogel structure. In some embodiments, diffusion of the target virus and the uniform distribution of nanoprobes in the biogel structure are important factors determining the detection speed and sensitivity. The particle aggregation limits detection performance. Given that the plasmonic nanoprobes are evenly distributed in the photonic-biogel structure, it is expected that a single peak appears around 550 nm at which each plasmonic-nanoprobe exhibits strong extinction as shown in FIG. 6a. To optimize the photonic-biogel structure, the density of the biogel (R_{m-c}) and population of the plasmonic nanoprobes (d_{prob}) in the structure are controlled. For the change in R_{m-c}, the ratio between the gel precursor and cross-linking agent is changed from 0.5 to 2.0. In some embodiments, different populations of plasmonic nanoprobes are represented by the corresponding optical density (OD) from 0.05 to 5.0. After the fabrication of the photonic-biogel, the optical properties of the samples were investigated by acquiring images and ultraviolet (UV)-visible (Vis) spectra of the samples (FIG. 7a). At the highest OD, the photonic-biogel exhibits dark red or wine color. Its transparency increased with the decrease in the OD. The photonic-biogel with the highest OD and lowest R_{m-c} was dark red, while the biogel with the lowest OD and highest R_{m-c} was red. The color distribution in the area for each sample (diameter, d×height, h=0.5×0.5 cm²) is also non-uniform in all samples. Subsequently, the optical properties of the samples were characterized using a UV-Vis spectrometer (FIG. 7b). In all the cases, the height of the spectrum decreased with the decrease in OD regardless of R_{m-c}. In particular, when OD was 2.0, a strong peak shoulder in the infrared (IR) range is observed, attributed to particle aggregation in the photonic-biogel. Although the IR peak shoulder became lower with the decrease in OD, the strong extinction around 550 nm, which represents a single nanoprobe, was not observed. These results indicate that the nanoprobes in the photonic-biogel structures may not be optimally dispersed in the gel structure.

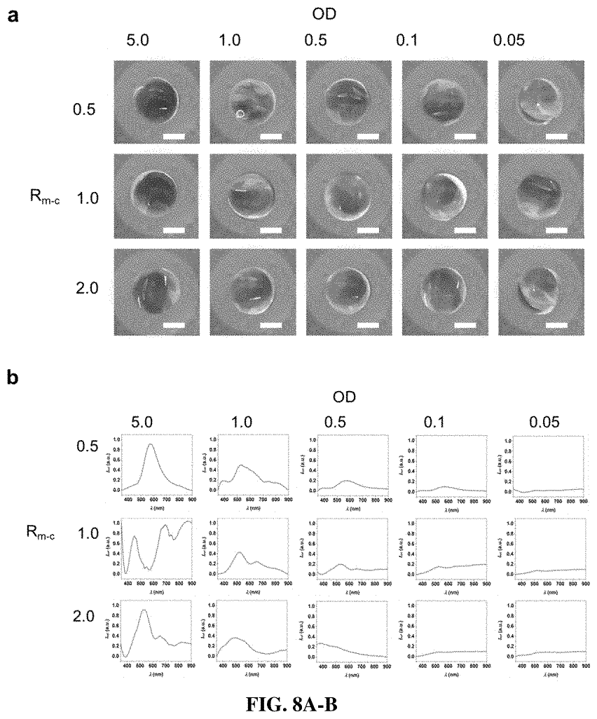

To improve the dispersion of the nanoprobe in the photonic-biogel, the co-polymerization process is modified by adding the nanoprobe after dissolving all agents for the biogel polymerization (FIG. 8).

FIG. 8 is a characterization of the photonic-biogel samples prepared by a b-staged co-polymerization.

FIG. 8a are photograph images of prepared photonic-biogel samples (scale bar=500 μm).

FIG. 8b are graphs of UV-Vis spectra of the photonic-biogel samples as a function of R_{m-c} of the biogel and OD of the plasmonic nanoprobes.

Additional samples were made with the same sample control parameters as before; OD of the nanoprobes and R_{m-c}. Similar trends in the color change and optical properties to those of the previous sample. However, at R_{m-c}=0.5, the shoulders around the IR region were not observed, while the higher R_{m-c} led to the IR shoulders. In particular, when OD was 2.0, a strong single extinction peak was observed at 550 nm. This indicates that nanoprobes with a larger density were uniformly distributed in the biogel with minimal aggregation.

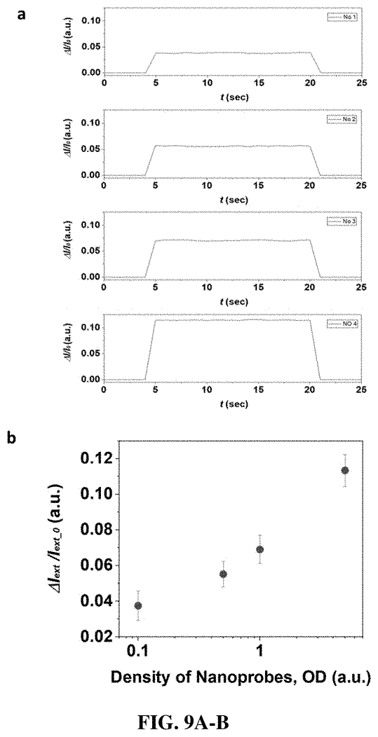

In addition, changes in optical properties of the sample were validated using the constructed optoelectronic detection system (FIG. 9).

FIG. 9 illustrates validation of the photonic-biogel integrated detection system.

FIG. 9a are graphs of measured photoresponse of the photonic-biogel samples as a function of OD of the plasmonic nanoprobe in the photonic-biogel under light off/on/off condition.

FIG. 9b illustrates quantified photocurrent variation as a function of OD of the plasmonic nanoprobe in the photonic-biogel.

With the samples at R_{m-c}=0.5, ΔI/I_₀ was measured as a function of d_{prob} using the complementary metal-oxide-semiconductor photodetector in the detection system (λ=532 nm). The measured ΔI/I_₀ linearly increased with the density of nanoprobes. This trend indicates that the nanoprobes in the biogel were minimally aggregated at R_{m-c}=0.5.

### Photonic Biogel

In some aspects, provided herein is a photonic biogel. In some embodiments, provided herein is a photonic biogel for use in spectroscopic detection of an airborne pathogen. In some embodiments, the photonic biogel can be incorporated into an air monitoring system described herein. For example, in some embodiments, the biochip 200 includes the photonic biogel 204 positioned within the trapping chamber 208. In other embodiments, the photonic biogel can be used in isolation, such as for spectroscopic detection of airborne pathogen(s).

In some embodiments, the photonic biogel (e.g. the photonic biogel 204) includes a plurality of nanoprobes distributed within a biogel. The nanoprobes distributed within the biogel are also referred to herein as “plasmonic nanoprobes”. In some embodiments, the photonic biogel comprises a cross-linked material. For example, in some embodiments the photonic biogel comprises cross-linked agarose. In some embodiments, the degree of cross-linking may be modified to avoid aggregation of the nanoprobes within the biogel. The degree of cross-linking within the biogel may also be referred to herein as the “density” of the biogel. A biogel of higher density will have a higher degree of crosslinking than a less dense biogel.

In some embodiments, the cross-linked material comprises a gel precursor and a cross-linking agent. In some embodiments, the degree of cross-linking (e.g. the density of the biogel) may be modified by controlling the ratio of gel precursor (e.g. agarose) to the cross linking agent. In some embodiments, the ratio of gel precursor to crosslinking agent may be about 0.5 (e.g. about 1 part gel precursor to about 2 parts cross-linking agent), about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0 (e.g. about 2 parts gel precursor to about 1 part cross linking agent) by weight (w/w). Accordingly, the “density” of the biogel (e.g. represented by the ratio of the gel precursor to the cross-linking agent) may be about 0.5 to about 2 (e.g. about 0.5, about 0.6, about 0.7, about 0.8, about 0.9, about 1.0, about 1.1, about 1.2, about 1.3, about 1.4, about 1.5, about 1.6, about 1.7, about 1.8, about 1.9, or about 2.0_

In some embodiments, the amount and distribution of the plasmonic nanoprobes within the biogel impacts the ability of the photonic biogel and systems comprising the same to accurately detect airborne pathogens. The amount and distribution of the plasmonic nanoprobes within the biogel can be indicated by the optical density of the plasmonic nanoprobes within the biogel. In some embodiments, the plurality of the plasmonic nanoprobes have an optical density within the biogel of about 0.05 to about 5.0. In some embodiments, the plurality of plasmonic nanoprobes have an optical density within the biogel of about 2.0. In some embodiments, the ratio of the gel precursor to the cross linking agent is about 0.5 (w/w) and the plurality of plasmonic nanoprobes have an optical density within the biogel of about 2.0.

In some embodiments, the plurality of nanoprobes comprise gold nanoparticles. In some embodiments, the gold nanoparticles are functionalized with a capture moiety. Various functionalization methods may be used, depending on the capture moiety. For example, in some embodiments the gold nanoparticles may be functionalized with an antibody by attachment of a suitable linker, such as a —COOH linker, to the surface of the gold nanoparticle. Such a linker may bind to the antibody. Any suitable capture moiety may be used. For example, the capture moiety may comprise an antibody or fragment thereof, an aptamer, a polyethylene glycol, a peptide, a protein, a nucleotide, a polynucleotide, and the like.

The capture moiety may be selected based upon the desired airborne particle to be detected using a system as described herein. Given that the disclosed photonic biogels and systems are useful for detecting a wide breadth of pathogens, it will be appreciated that a variety of capture moieties can be used. In some embodiments, the capture moiety is an antibody. The antibody may be selected based upon the desired airborne particle to be detected. For example, in some embodiments the airborne pathogen is a virus. In some embodiments, the airborne pathogen is a respiratory virus, including but not limited to a respiratory syncytial virus, a parainfluenza virus, a metapneumovirus, a rhinovirus, a respiratory adenovirus, a coronavirus, a severe acute respiratory syndrome (SARS) coronavirus, a bocavirus, a parvovirus, or an influenza virus. In some embodiments, the virus is SARS-CoV-2. For viruses, antibody capture moieties may be preferable. For example, for detection of SARS-CoV-2, the antibody may be an antibody to the spike protein (e.g. S-protein) of SARS-CoV-2.

In some embodiments, the capture moiety comprises an amino acid. For example, for detection of airborne bacteria a suitable capture moiety may comprise an amino acid. In some embodiments, the airborne pathogen is a gram-negative bacteria. In some embodiments, the airborne pathogen is a gram-negative bacteria and the capture moiety comprises an amino acid. In some embodiments, the airborne pathogen is a gram-negative bacteria and the capture moiety comprises a neutral amino acid (e.g. from cysteine, glutamine, asparagine, threonine, or serine molecule) In some embodiments, the capture moiety is a cysteine molecule.

In the illustrated embodiment, the trapping chamber 308 is cylindrical and includes a diameter 212 that is equal to a light source diameter (e.g., a laser beam diameter).

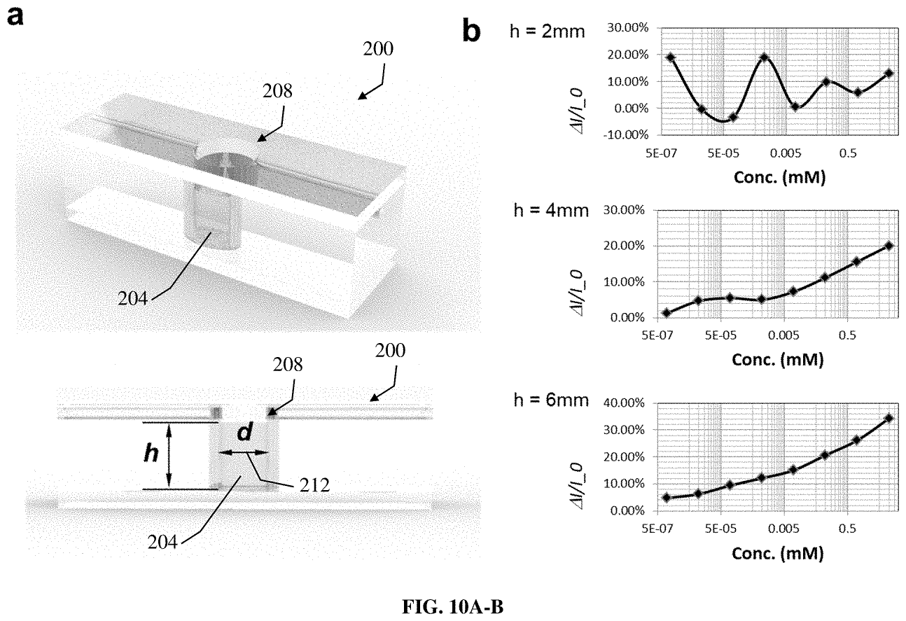

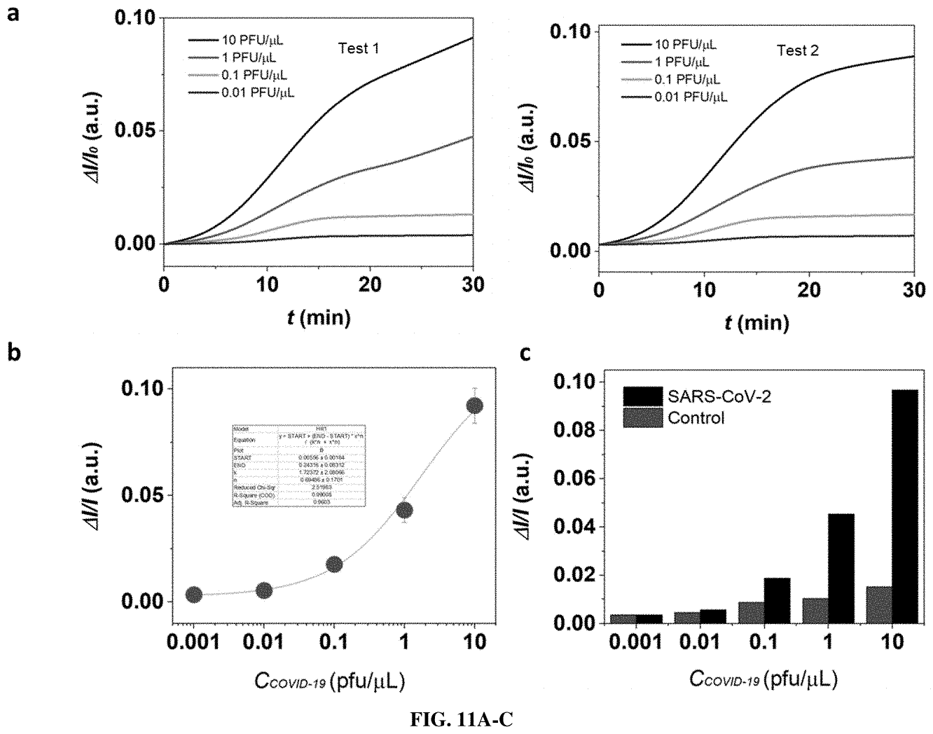

FIG. 10 illustrates a microfluidic biochip with integration of a photonic-biogel.

FIG. 10A illustrates the microfluidic biochip design with a cylindrical shaped chamber having a diameter, d (i.e., the diameter 212) and a height, h.

FIG. 10b illustrates the effect of the geometry change on the detection sensitivity; calibration curves for methylene blue as a function of the chamber height from 2 and 4 mm.

When a photonic-biogel is embedded into the micro-optofluidic chip, the light transmission and reflection can differ as the gel acts as a liquid layer in the chip, resulting in reduced detection sensitivity. To incorporate the photonic-biogel into the micro-optofluidic chip, the effect of the geometry change of the detection chamber on the sensitivity using a standard liquid sample is estimated (FIG. 10b). Considering that the detection principle is based on the measurement of the change in optical transmission as a function of the molecular concentration in the chamber, the chamber shape and height are modified in the illustrated embodiment of FIG. 10a. To minimize the scattering effect in the chamber, the width of the chamber was reduced to match the size of the laser. The chamber height (h) is strongly associated with the optical transmittance. FIG. 10b illustrates the photocurrent variation (ΔI/I_₀) as a function of the concentration of the standard test molecule (methylene blue)(C_{MB})) at different heights of 2, 4, and 6 mm. When the height was 2 mm, ΔI/I_₀ exhibited a high signal fluctuation as a function of the concentration in the range of 10⁻⁶ to 10⁰ M. With the increase in height, it stabilized and ΔI/I_₀ was highly sensitive. For example, at h=6 mm, ΔI/I_₀ increased with C_{MB} in the same concentration range. According to the calibration curves, the estimated LODs were 8.0×10⁻¹, 1.3×10⁻³, and 7.1×10⁻⁵ M at h=2, 4, and 6 mm, respectively.

### Virus Detection

The photonic biogel described herein or the system described herein find use in a variety of methods. In some embodiments, the photonic biogel or the system described herein is used in a method of spectroscopically detecting an airborne pathogen. In some embodiments, methods of spectroscopically detecting an airborne pathogen comprise obtaining a one or more measurements of optical transmission of the photonic biogel. In some embodiments, methods of spectroscopically detecting an airborne pathogen comprise obtaining a baseline optical transmission value of the photonic biogel, exposing the photonic biogel to an environment having or suspected of having the airborne pathogen, and obtaining a second optical transmission value of the photonic biogel following exposure to the environment. In some embodiments, a change in the second optical transmission value compared to the baseline value indicates that the airborne pathogen is present in the environment. In some embodiments, a decrease in the second optical transmission value compared to the baseline optical transmission value indicates that the airborne pathogen is present in the environment. Accordingly, the photonic biogels, systems, and methods described herein can be used to detect one or more airborne pathogens in an environment without the need to perform downstream assays. Rather, the plasmonic nanoprobes themselves permit spectroscopic means to be used to evaluate the biogel post-exposure to an environment in order to determine whether an airborne pathogen (e.g. a virus, a bacteria, etc.) is present (or absent) in the environment. The photonic biogels and systems described herein can thus be useful for monitoring air quality and safety, which can be useful for protecting subjects (e.g. humans) from being exposed to airborne pathogens in various locations, including hospitals, schools, workplaces, and the like.

FIG. 11 illustrates detection performance of SARS-CoV-2 particles in the photonic-biogel.

FIG. 11a is a graph of dynamic binding curves of SARS-CoV-2 particles at different concentrations from 0.001 to 10 pfu/μL,

FIG. 11b is a graph of a calibration curve for SARS-CoV-2 in the photonic-biogel.

FIG. 11c is a bar chart of the biosensor signal for PBS solutions spiked with SARS-CoV-2 particles and control particles (SiO₂ nanoparticle) at various concentrations.

Using the optimized photonic-biogel and micro-optofluidic biochip, detection of SARS-CoV-2 particles is performed (FIG. 11). After injecting a virus sample solution on top of the biogel (R_{m-c}=0.5, OD=2.0), the dynamic ΔI/I_₀ is measured as a function of the SARS-CoV-2 concentration (C_{SARS-CoV-2}) in the range of 0.01 to 10 pfu/μL. The variation in the concentration of the SARS-CoV-2 particles is expected changes the spectral intensity of the extinction peak of the nanoprobe and virus according to the wavelength of the light source. The increase in the C_{SARS-CoV-2} led to a lower optical transmission, leading to a decrease in the photocurrent of the device. At λ=780 nm, ΔI/I_₀ increased from 0 to 0.06 with the increase in C_{SARS-CoV-2} from 0.01 to 10 pfu/μL. The ΔI/I_₀—time curves in the measured range of C_{SARS-CoV-2} reached a steady state in approximately 30 min. The standard calibration curve shows that the device provides a sensitive detection with a large (104) dynamic range. In addition, the LOD (0.13 pfu/μL) for the biogel-integrated biochip was determined using the obtained calibration curve. All LOD values are presented by 3σ/kₛₗₒₚₑ, where σ and kₛₗₒₚₑ are the standard deviation of the background signal measured from a blank control and regression slope of the calibration curve, respectively.

### Bacteria Detection

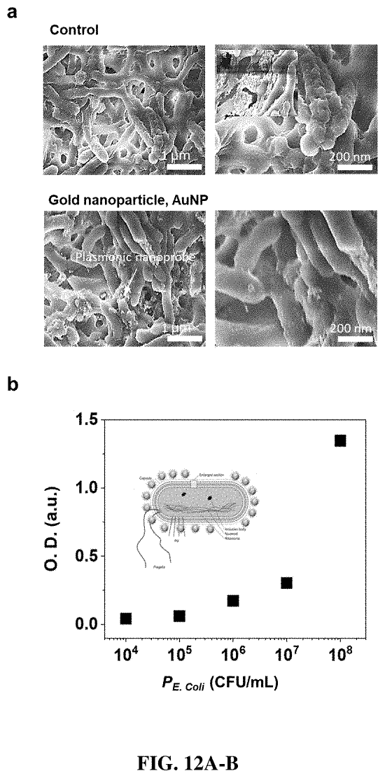

FIG. 12 illustrates detection performance of E. coli particles.

FIG. 12a are SEM images of E. coli samples with control particle (conventional AuNPs) and synthesized plasmonic nanoprobes.

FIG. 12b is a graph of Measured OD for the mixture of the plasmonic nanoprobes and E. coli as a function of E. coli density from 10⁴ to 10⁸ cfu/mL.

The ability of the biosensor platform, disclosed herein, to detect bacterial particles is also investigated. Firstly, a plasmonic-nanoprobe is designed and synthesized to detect toxic bacterial particles. Considering the membrane structure of toxic bacterial particles (gram-negative bacteria), a charge matching method was employed. The surface charge of the bacterial membrane revealing a highly negative charge (e.g., approximately −40 mV) is matched to the new plasmonic nanoprobe existing strong positive charge. For the detection test, E. coli was chosen as a representative bacteria model, which is a gram-negative bacteria. For the plasmonic nanoprobe, cysteine molecules were functionalized on AuNPs surface. Then, the mixture of E. coli and the plasmonic nanoprobes were prepared by incubating for 30 minute, and scanning electron microscope (SEM) images were obtained to compare with the control sample (another mixture of E. coli and conventional AuNP particles) (FIG. 12a). The plasmonic nanoprobes are well distributed on the surface of E. coli sample, while any visible interaction between conventional AuNP and E. coli is not observed. Furthermore, this interaction between plasmonic nanoprobes and the E. coli sample enabled to the quantification of E. coli particles in the sample without culturing and sample treatment. FIG. 12b illustrates a graph showing the measured OD vs E. coli concentration revealed a calibration curve for E. coli (LOD=approximately 5×10⁴ cfu/mL). These results indicate that the synthesized plasmonic nanoprobe holds a capability of E. coli detection with high sensitivity.

Air Quality Monitoring System with a Plurality of Detection Channels

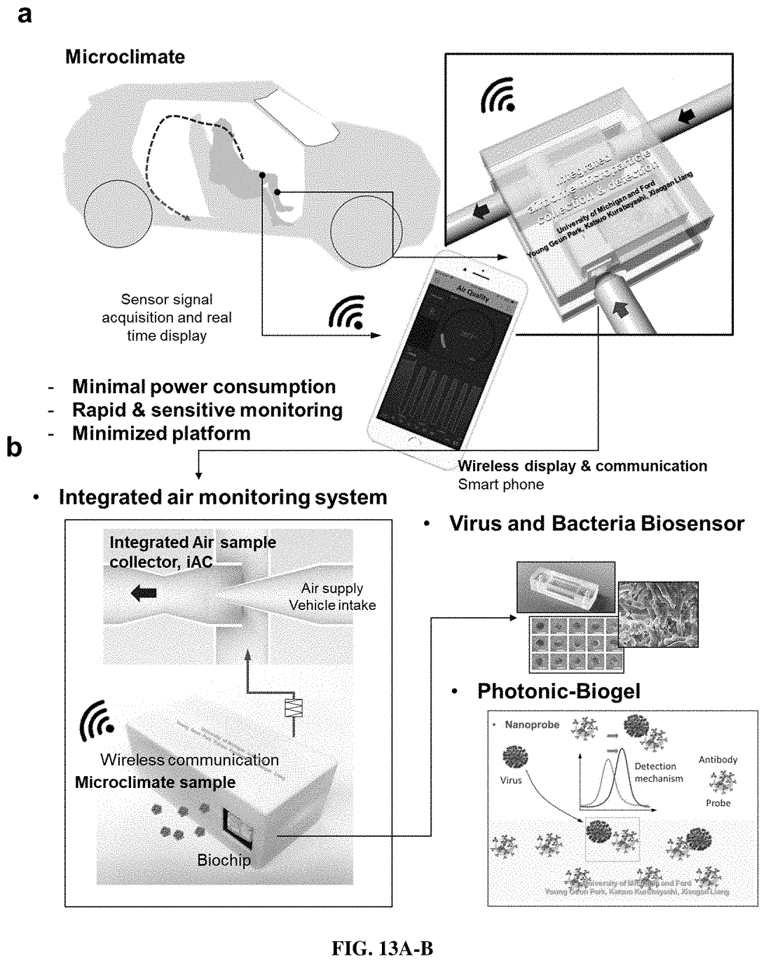

With reference to FIG. 13, a smartphone-based integrated microsystem for real-time on-site collection and detection of indoor airborn microparticles with high sensitivity is illustrated. The system collects airborne microparticles using the Venturi effect, which generates a consistent negative pressure in the air by high-speed airflow. The negative pressure draws microparticles to the collection unit of the system regardless of their concentration in the air sample. The optimal design and operating conditions of the collection unit determined by finite element analysis (FEA) allow us to collect a wide range of airborne particles. The collection unit is integrated with a detection unit of the system. The detection unit comprises a microfluidic particle trapping chamber and a complementary metal-oxide-semiconductor (CMOS) photodetector. In some embodiments, the entire system is operated by smartphone-based communication with an app created on, for example, the Blynk IoT Platform. As detailed herein, the system of FIG. 13 is an airborne microparticle monitoring system with high sensitivity, fast speed, and simple operating capabilities is imperative for a micro-climate setting.

To achieve direct, rapid, and sensitive in-situ detection of airborne virus particles, the biochip disclosed herein is configured for compact and portable biosensing. The air-flow microfluidic biochip incorporates a biofunctional gold nanoparticle (AuNP)-embedded hydrogel layer called the “photonic-biogel,” and a miniature micro-optics architecture. Here, the biofunctional AuNPs serve as plasmonic nanoprobes. The interaction of airborne particles and nanoprobes causes an optical transmission shift of the hydrogel layer. The transmission shift is quantitatively correlated with the virus population in the air. Achieving the high density and uniform distribution of plasmonic nanoprobes in the photonic biogel is important to facilitate rapid virus particle diffusion and analyte-nanoparticle interactions. This allows the system to respond rapidly and sensitively. In addition, optimizing the microfluidic biochip design enhances the photodetector's sensitivity to a transmission change.

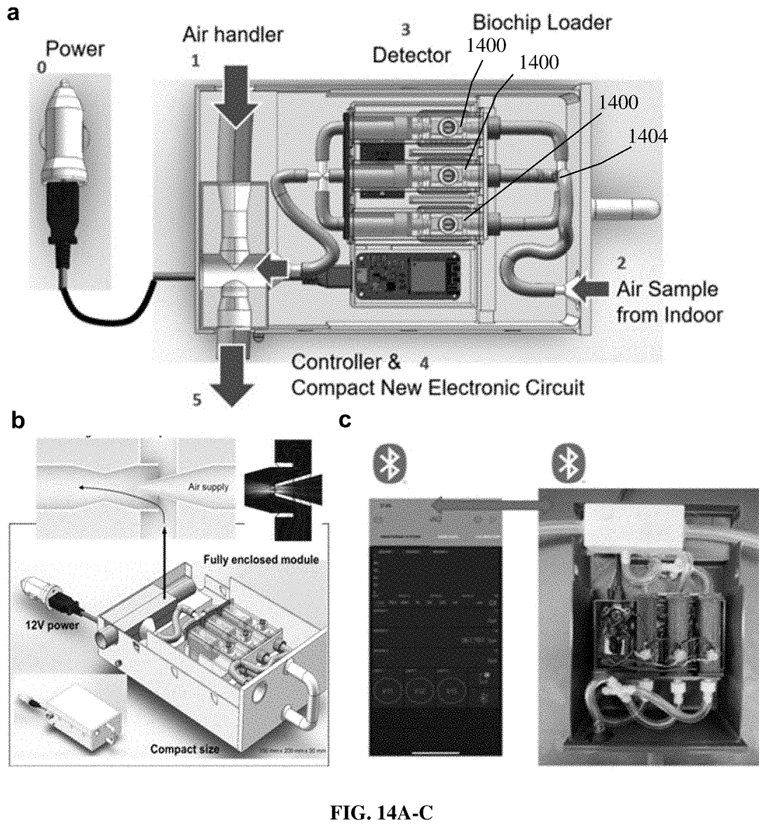

With reference to FIG. 14, a fully integrated air quality monitoring system is illustrated with three detection channels 1400. The modular design of the system allows users to repeat the measurement by conveniently replacing the biochip. The airborne particle detection performance of the system is improved. The impact of optimized photonic-biogel synthesis and light source selection on the sensitivity of virus particle detection is demonstrated herein. In some embodiments, real-time wireless data acquisition and transmission for the system using a standalone IOS and the Android app. In some embodiments, the photocurrent signal varies with the concentration of virus-contained aerosols. In the illustrated embodiment, the system can collect enough aerosols for detection, generate consistent signals in a short time period, and alert the presence of the virus.

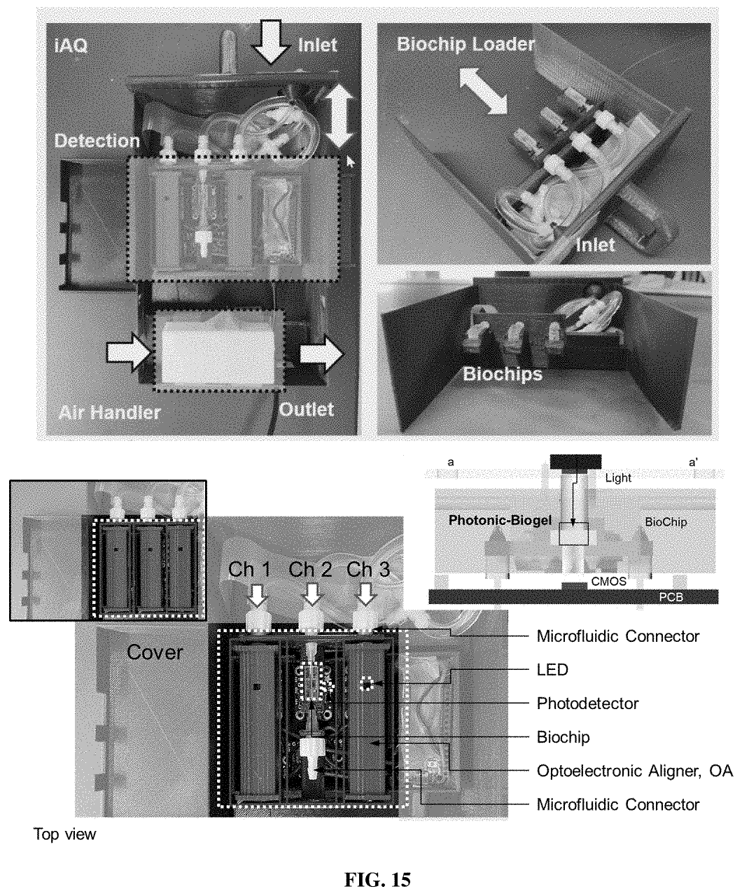

With reference to FIG. 15, the integrated detection system comprises a detection unit, an air handler, and an inlet and outlet. The biochips are loaded into the manual sample loader. The detection unit contains three channels of detection parts. Individual detection parts are assembled with two microfluidic connectors, an LED, a photodetector, a biochip, an optofluidic aligner, and a photodetector.

With continued reference to FIG. 15, the integrated air quality monitoring system includes three sections: (1) an air sample collection or handler section, (2) a detection section including a light source, a biochip, and a photodetector, and (3) a biochip loader section. In some embodiments, the parts are fabricated with additive manufacturing (e.g., 3D printing). In some embodiments, components are fabricated with polylactic acid (PLA). In some embodiments, the tubes used for airflow collection were TYRON R-3603 laboratory and vacuum tubing. In some embodiments, the CMOS photodetector (e.g., Adafruit TSL2591 light sensor) is connected to an esp32 (Adafruit esp32 microcontroller) via I²C communication protocol.

In some embodiments, the operation voltage of the photodetector and the logic voltage of the esp32 microcontroller are both 3.3 V, and therefore, they are directly connected using, for example, jumper wires soldered on each pin. In some embodiments, because the smartphone app communicates with the device only through Bluetooth 2.0 serial, for power saving, in esp32 microcontroller settings, all wireless communication protocols other than Bluetooth classic such as Wi-Fi and Bluetooth Low Energy (BLE) are turned off. In some embodiments, the light sources (e.g., Adafruit LED Sequins, Ruby Red, I=50 mcd, λₚ=632 nm) were soldered onto microcontrollers and attached to the detection channels using hot glue.

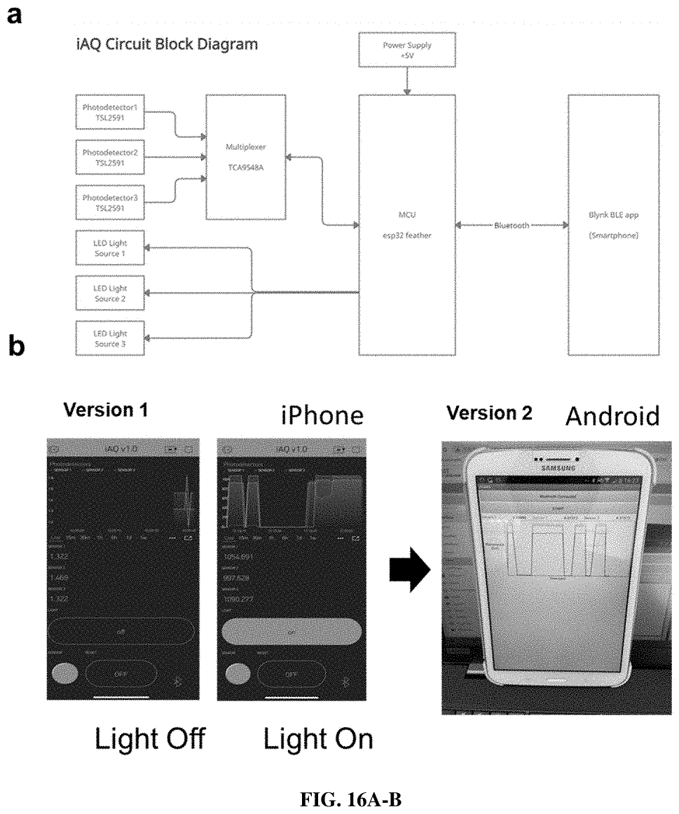

Regarding the application software, versions of application software were created for a standalone IOS and Android app for the Internet of Things (IoT) operation of the integrated air quality monitoring system (FIG. 16). The application software allows the integrated system to be controlled and real-time data from the sensors to be collected.

### EXAMPLES AND METHODS

Materials and Chemical-/Biological-Agents

Polystyrene (PS) particles (monosized standard spherical particle: 1 μm in diameter; refractive index 1.59; density 1.06 g/cm³; Duke Scientific Corporation, Palo Alto, Calif., USA) was used to evaluate physical particle collection efficiency. The E. coli, Bacillus subtilis, Micrococcus luteus, and Staphylococcus were purchased from Carolina biological supply (Burlington, N.C., USA).

Gold nanoparticle (AuNPs, d=40 nm) were purchased from Tedpella. 10-Carboxy-1-decanethiol (C-10), agarose powder, Tris-Acetate-EDTA (TAE), and albumin, from bovine serum (BSA), were purchased from Sigma Aldrich. 1-ethyl-3-[3-dimethylaminopropyl]carbodiimide (EDC) and/N-hydroxysuccinimide (NHS) were purchased from ThermoFirscher Co. Polydimethylsiloxane (PDMS) elastomer and curing agent were purchased from Coring. Nano pure deionized (DI) water (18.1 MΩ-cm) produced internally. For experiments using the virus, heat-inactivated SARS-related coronavirus 2 was purchased from ATCC. SARS-CoV-2 spike antigen protein (40591-V08H) and SARS-CoV-2 spike antibody (40150-R007) were purchased from Sino Biological, Inc., China.

Finite Element Analysis

To predict the effect of the main design parameters impacting the performance of the airborne particle collection device and the biochip, FEA (COMSOL Multiphysics software) was conducted to obtain the particle trajectory, pressure distribution and velocity field in the airborne particle collection device and the biochip as a function of the geometric parameters. The fluid drag force was estimated from the Khan and Richardson force.

Fabrication of Airborne Particle Collection Device

The airborne particle collection device consisted of three parts of air supply inlet, air sampling port, and channel body including outlet, designed on AutoCAD. Using a 3D printer (Prusa research, Prusa 13 MK3S) and a mechanical cutter, each part was obtained with polylactic acid (PLA). After the parts of the airborne particle collection device were printed, they were assembled and bonded together using acrylic cement (SCI-GRIP).

Fabrication of the Biochip

In some embodiments, the biochip consists of three layers: i) a top layer containing an inlet, an optical window, and an outlet, ii) a middle layer functioning as a micro chamber, and iii) a bottom layer playing roles of an adhesive layer and an optical window. The biochip was assembled the three layers cut from poly(methyl methacrylate) (PMMA) by a laser cutter (Versa Laser, ULS 4.60) by PSA (3M, 268L) films. Before the assembly step, a PSA layer was attached onto the channel bottom. To ensure the integrity of the assembled device, after assembly, the device was pressed using a hydraulic presses (Atlas Manual Press) under 7.5 tons for 30 min.

In some embodiments, the biochip consists of five layers: (1) a bottom layer, which plays the role of an adhesive layer and an optical window, (2) a container layer that provides space for the agarose gel and nanoprobe consisting of AuNPs and antibodies, (3) a middle layer functioning as a micro-reaction chamber in which particles in airflow are collected and accumulated; (4) an in/out layer, which contains the inlet and outlet for airflow; and (5) a top layer functioning as an optical window and top enclosing layer. In some embodiments, the biochip layers are cut out of methyl methacrylate (acrylic) sheets using a laser cutter (Universal Laser Systems X2-600). In some embodiments, the layers are assembled together using, for example, Gorilla Super Glue. In some embodiments, the layers are clamped together for 5 minutes to ensure the integrity of the assembled biochips. After the fabrication of all the layers, 100 μL of 5 mg/mL agarose gel is injected into the reaction chamber of the biochip, and thereafter, 10 μL of AuNP linked with antibodies are placed on the surface of the agarose gel. In some embodiments, electrical tape is wrapped around the biochip to achieve an enclosed reaction environment.

Synthesis and Characterization of Nanoprobes

To prepare the nanoprobes, AuNP were centrifuged three times at 5,000 rpm for 10 min and washed in D.I. water to remove excessive structure direction agents in the solutions. After preparation of the purified AuNP colloidal solution, functionalization of thiolated alkane 10-Carboxy-1-decanethiol (HS—(CH₂)10-COOH) using a self-assembly method followed. At first, AuNP colloidal solution was incubated in 1 mM of thiolated alkane 10-Carboxy-1-decanethiol (HS—(CH₂)₁₀—COOH) overnight. Then, the formed carboxylic group (—COOH) on the AuNP surface enabled the attachment of a linker to the antibody. The antibody linking was performed by antibody binding to the —COOH functional group through EDC/NHS coupling chemistry. After washing the —COOH formed AuNP, the treated AuNP were loaded into a mixture of 0.4 M EDC and 0.1 M NHS at a 1:1 volume ratio in a 0.1 M EDC solution to activate the AuNP. Then, to attach the antibody, diluted antibodies 10 μg/mL were prepared in 1× buffer solution. The prepared antibody solution was loaded into the micro-tube and incubated for 60 minutes. To suppress the non-specific binding on the detection surface, the prepared Anti-AuNP conjugates were treated with 1% BSA in 1×PBS in blocking buffer and incubated the whole system for 20 minutes. Before detecting S-protein or SARS-CoV-2 particles, the Anti-AuNP particles were thoroughly washed three times to remove any excessive solutions or molecules using 20 μL of 1×PBS. In addition, a spectrum of nanoplasmonic colorimetry is acquired using a UV-VIS spectrometer (Agilent 8453 G1103A Spectrophotometer).

Synthesis and Characterization of Photonic-Biogel

Agarose powder was added slowly to water to make a 0.5, 1, and 2 wt % aqueous agarose solutions with 1×TAE under vigorous stirring at room temperature, and then heated to boiling for 1 minute, acquiring a clear solution. The solution was poured gently into containers and incubated for 20 minutes. After 10 minutes, in the middle of the cooling step, the prepared plasmonic nanoprobe was injected into the solution. The containers were then covered with parafilm and left overnight. The prepared gel was cut with a scalpel into small pieces in water. Because PBS solution was used as the solvent for the biosensing, the agarose gel underwent solvent exchange. The gel pieces were transferred from a water solution to a mixture of water and PBS (2:1 by volume) solution for at least 6 hours, followed by transfer to a mixture of water and PBS (1:2 by volume) solution and finally placed into PBS. The agarose gel pieces were stored in the PBS ready for sample characterization. Then, the optical properties of the samples were measured by a UV-Vis spectrometer (Agilent 8453). Morphologies of the bacterial samples were analyzed by SEM images.

In some embodiments, the photonic-biogel is synthesized by adding agarose powder slowly to water, to prepare 2 wt % aqueous agarose solutions with 5XTAE under vigorous stirring at room temperature, and then heated to boiling for 1 min to obtain a clear solution. The solution was gently poured into the containers. After 10 min, the prepared plasmonic nanoprobe was injected into the solution, in the middle of the cooling step. The containers were then covered with parafilm and left overnight.

Biochip with Integration of Photonic-Biogel

The microfluidic biochip was assembled in three layers; i) a top layer containing an inlet, an optical window, and an outlet, ii) a middle layer functioning as a micro chamber, and iii) a bottom layer playing roles of an optical window by PSA (3M, 268L) films. The three layers were made of poly(methyl methacrylate) (PMMA) and cut by a laser cutter (Versa Laser, ULS 4.60). To integrate the photonic-biogel into the microfluidic chip, in the middle of the photonic-biogel preparation, the pre-cured solution was poured into the partially assembled microfluidic biochip without top window layers. After the curing step, a solvent exchange step followed to replace water in the gel with PBS solution and the top layer was assembled. To ensure the integrity of the assembled device, after assembly, the device was pressed using a hydraulic presses (Atlas Manual Press) under 7.5 tons for 30 minutes.

Integrated Detector Device

For the integrated detection, firstly, a printed circuit board (PCB) (W×L×H=64.7 mm×31.2 mm×1 mm) was integrated with an Arduino Nano (WYPH, Arduino Nano), a microcontroller (MCU, ATMEGA328P), a commercial CMOS photodetector (ams, TSL2591), a Bluetooth BLE (DSD Tech, HM-10), a light source (Luckylight, LL-S150 W-W2-1C, I=350 mcd), and Li-ion battery (2500 mA and 3.7 V). The commercial CMOS photodetector (AMS, TSL2591) was connected to the Arduino Nano with an I²C communication protocol. The operation voltage of the photodetector and logic voltage of Arduino Nano are 3.3 V and 5 V, respectively. To compensate such discrepancy and make a stable I²C communication, a level shifter circuit using two FET transistors (On Semiconductors, BSS138) was utlized. A voltage regulator (Microchip Technology, MIC5225) converts the applied voltage (9 V) to 3.3 V.

The logic levels of data (SDA) and clock (SCL) lines in the I²C communication are biased as “High” at the idle condition. In order to wirelessly deliver sensor signal to outside iOS application, the Bluetooth BLE (DSD Tech, HM-10) was incorporated into the PCB. The operation voltage of each device was provided by a voltage regulator (Microchip Technology, MIC5225) which converts the applied voltage (9 V) to 3.3 V. Then, the microcontroller (MCU, ATMEGA328P) was combined with the as-fabricated PCB to control peripheral devices such as the CMOS photodetector, the voltage regulator, and the Bluetooth BLE (DSD Tech, HM-10). Finally, the integrated system with a light source (Luckylight, LL-S150 W-W2-1C, I=350 mcd) was enclosed by a package box printed out from a 3D printer (Prusa research, Prusa 13 MK3S). For the data communication and display, Blynk including an application software (https://blvnk.io/) was employed to construct IoT environment. A BLE terminal for a smartphone (Apple, iphone 8) was utilized as application software for remote data communication and display.

Bacterial Particle Preparation

All reactors were sterilized by autoclaving at 120° C. for 900 s. Bacterial particles were cultured to log phase at 37° C. with shaking of 200 rpm, and harvested by centrifugation at 900 g, washed twice with deionized (DI) water. Then suspended in DI water to ˜10⁶ CFU/mL and concentrated them by congregation at 900 g for 10 minutes.

Airborne Particle Detection Test

The aerosol generator, the biochip, and the airborne particle collection device were connected by PTFE tubing. By adjusting the concentration of microparticle suspension in the aerosol generator (BEIBERSI, BSW-2A, China), the density of airborne particle in the airflow sample was controlled. By turning on the aerosol generator, uniform sized airborne particle distribution in the air was generated. When the air sample containing the defined concentration of airborne particle flew into the biochip, the airborne particles were captured into the biochip. The optical density change induced by the captured airborne particle was measured by the underneath CMOS.

In some embodiments, the virus stocks were aliquoted and stored at −80° C. Stock volumes were either used for direct experimentation or diluted in TE Buffer. Serially diluted SARS-CoV-2 heat-inactivated viruses in TE buffer were spiked directly into PBS solution ranged from 0.001 PFU/μL to 10 PFU/μL. For the photonic-biogel performance test, after mixing the nanoprobes and the prepared virus solution, it was loaded into the biochip chamber. Subsequently, the photocurrent signal change from photonic-biogel was measured using the integrated CMOS photodetector device.

Virus Detection Test

The detection of the virus is based on the change in light transmission induced by the binding between the virus and the plasmonic nanoprobe. When combined with the S protein on the virus, the plasmonic nanoprobe (AuNP with antibodies) leads to an LSPR frequency and intensity change. These changes were quantified by measuring the absorbance at approximately 650 nm. Light absorbance is defined as the logarithm of the ratio of incidence to the transmitted radiant power through a sample¹¹: A=ln(Iᵢₙ/Iₒᵤₜ), where A, Iᵢₙ, and Iₒᵤₜ are the absorbance, the light intensity resulting from the nanoprobe before detection, and the light intensity resulting from detection, respectively. This procedure was simplified to measure the variation of the photocurrent, that is, ΔI/I₀, where ΔI=I₀−I, I is the real-time measured light intensity, and I₀ is the initial light intensity from the nanoprobe before detection. Theoretically, the real-time photocurrent variation would gradually decrease at a decreasing rate until a steady state is reached, and the decreasing rate is affected by the concentration of the virus because it influences the reaction rate with antibodies on plasmonic nanoprobe. Therefore, after a certain time range of detection, the resulting photocurrent variation is translated into the concentration of virus particles in the surrounding environment, which means that once a variation in the light intensity has been computed, the coherent virus particle concentration can be found.

Experiment Setup

To detect SARS-CoV-2 in the air flow sample, a nebulizer (WH-2000) is used. The nebulizer allowed the creation of an aerosol environment that mimics the microclimate. To achieve uniform aerosol conditions, 3 mL of water and 60 μL of samples (water, SiO₂, or SARS-CoV-2) were loaded into the chamber. The nebulizer and integrated system were connected through a tube to inject the generated aerosols. In terms of the experimental procedure, once turned on, the created aerosol filled the chamber with “mist” of mixtures of water and sample particles. The negative pressure (ΔP=0.03 bar) generated by the air-hander (collection device) dragged the aerosols into the biochip, in which most of the particles accumulated, and thus caused the nanoprobes to interact with the airborne particles to form a reaction. Such a reaction would result in a change in the absorbance of the medium including the plasmonic nanoprobes and the agarose gel, and could be detected by the CMOS photodetector beneath the biochip.

Design and Construction of an Integrated Air Quality Monitoring System

As a fully integrated air quality monitoring system, the system simultaneously identifies several airborne species in a sample of air. The disclosed system has three detecting channels 1400 (FIGS. 13 and 14). A single incoming flow carrying airborne pathogen particles is separated into three equal channels by a flow splitter 1404 in the integrated system. There is a biochip with a photonic-biogel enabling bio specificity in each channel that can detect certain airborne particles in the airflow. In the illustrated embodiment, channels 1 and 2 detect viruses and bacteria, respectively. Channel 3 is a reference to ensure baseline. In some embodiments, to simultaneously identify other airborne pathogens in the air sample, more channels can be added in any quantity.

The application software disclosed herein assists the user with continuously monitoring and checking the level of raw data. In some embodiments, the application software enables a smartphone to control the detecting device and collect data in real time. In some embodiments, the software includes the following detailed functionalities: i) turning on/off the LED light, ii) collecting raw data from the photodetector, iii) displaying the obtained raw data without any data processing on the smartphone, and/or iv) transferring the collected raw data to a secondary location for further analysis.

As illustrated, the integrated system uses three detection units: an air handler, a biochip loader, and a detection unit. The detection unit comprises a biochip, a light-emitting diode (LED) as the light source, and a CMOS photodetector. The airborne particle trapping in the biochip leads to changes in the optical transmission. An optical transmission change is detected by the CMOS photodetector under light illumination from the top LED. The optical transmission change results in a change in photocurrent (I) through the integrated CMOS photodetector. The measured I represents the density of the trapped particles from the airflow collected by the airborne particle collection device. In detail, the operation involved (STEP i) supplying air to the air handler (airborne particle collection) device to generate negative pressure and induce the collection of air samples containing airborne microparticles from the microclimate, (STEP ii) flowing of the collected air sample into the biochip, (STEP iii) capturing airborne particles in the air sample into the biochip, (STEP iv) measuring a change in optical transmission under light illumination, (STEP v) transmitting the measured optical signal change to a smartphone, and (STEP vi) displaying the measured data by the installed application software.

After data acquisition, the biochip can be replaced with a new one for the next measurement. This permits the repeated use of the same CMOS photodetectors and LEDs. In some embodiments, the multiplexer (e.g., Adafruit TCA9548A) separates the addresses of the three photodetectors. This arrangement allowed the microcontroller unit to communicate with each of the photodetectors individually via the I2C channel. The microcontroller first recognized the address of the multiplexer and obtained a list of available sub-addresses that were connected to the photodetectors. Every time the microcontroller read the signal of the photodetector, it selected the sub-address of the target light sensor, proceeded the sensor reading and data processing programs, and then selected the next sub-address for repeating the operation cycle.

Validation of Air Flow and Optical Signal Stability

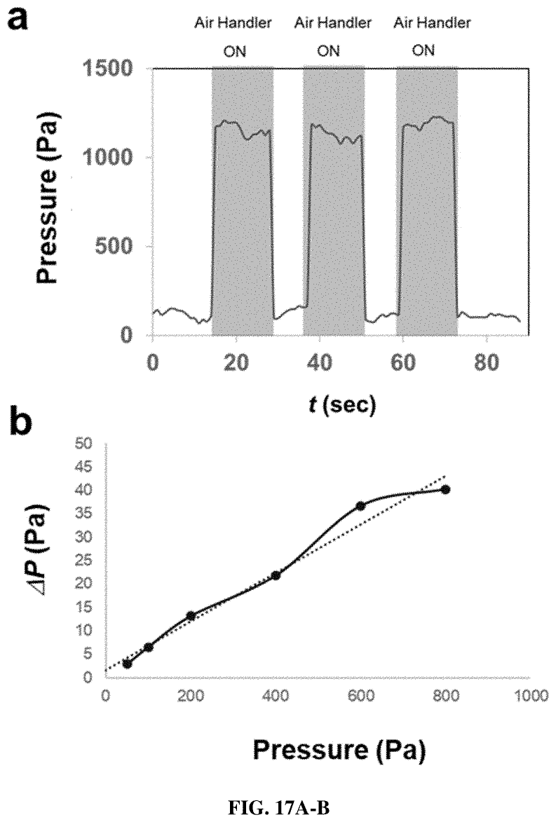

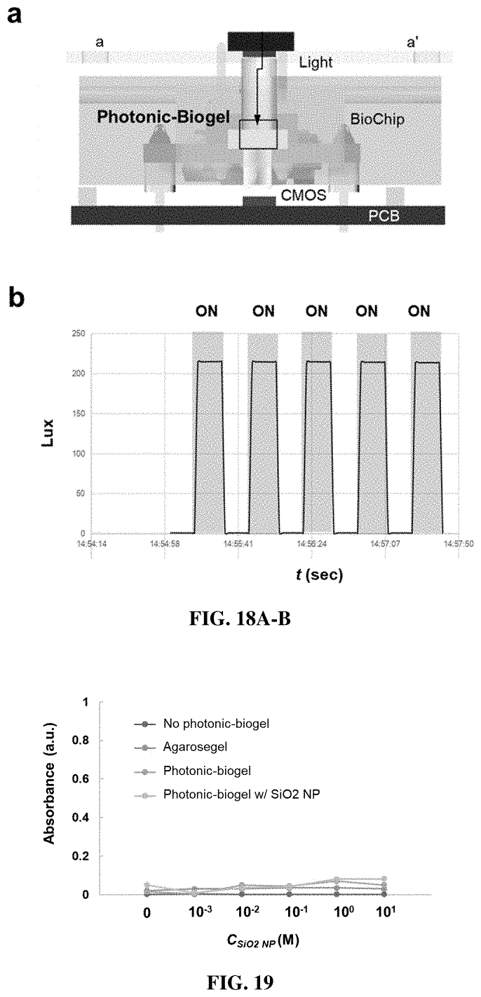

Using an integrated system of air quality monitoring, the proper air flow and optical signal stability is established. First, by turning on the air handler, the air flow generation in the integrated system (FIG. 17) was checked. Next, the pressure in the system is measured to ensure airflow occurred in the system. The on/off cycles of the air handler (P=0.015 Bar) are repeated and observed pressure variations between the air handler's ON and OFF states. The system pressure followed a linear trend with the air handler pressure. This trend indicates that the pressure in the system was tunable. These findings suggest that the integrated system accurately created airflow for the downstream measurement. Subsequently, the air sample is drawn through the system and the optical signal variation (ΔI/I₀) is directly measured. Then the optical signal stability is configured (FIG. 18). In some embodiments, a minimal change in ΔI/I₀ is observed with the varying density of the SiO₂ particles.

Baseline with Agarose Gel, AuNP, and Light Source

With reference to FIG. 19, after confirming the system stability, the baseline of the detection unit components, (e.g., the agarose gel, the photonic-biogel, and the light source) is examined. The use of SiO₂ nanoparticles provides a baseline for these components because they do not have any chemical/biological affinities with the antibodies on the prepared nanoprobes. After loading SiO₂ nanoparticles into the nebulizer, an airflow sample is generated containing the SiO₂ nanoparticles and the optical signal variation (ΔI/I₀) is measured with the concentration of SiO₂ particles varying from 10³ to 10⁸ particles/mL. A minimal change in ΔI/I₀ is observed with a change in the SiO₂ nanoparticle concentration. As such, the detection unit is insensitive to non-target airborne particles.

System Level Detection of Airborne Particles in Airflow

A system-level detection of airborne particles in an airflow sample is performed. First, an airflow sample is generated by loading a known concentration of airborne particles into the nebulizer. Thereafter, the air handler is turned on to generate a pressure drop between the inlet and outlet of the system. Finally, a valve between the nebulizer and the integrated detection system is opened to generate air flow containing airborne particles, which was followed by the measurement of the real-time signal variation. Multiple tests for various airborne particle concentrations are performed. The average sensor readings from the last 10 seconds were recorded and processed for photocurrent variations. The data collection frequency was set to be high enough to detect signal fluctuations.

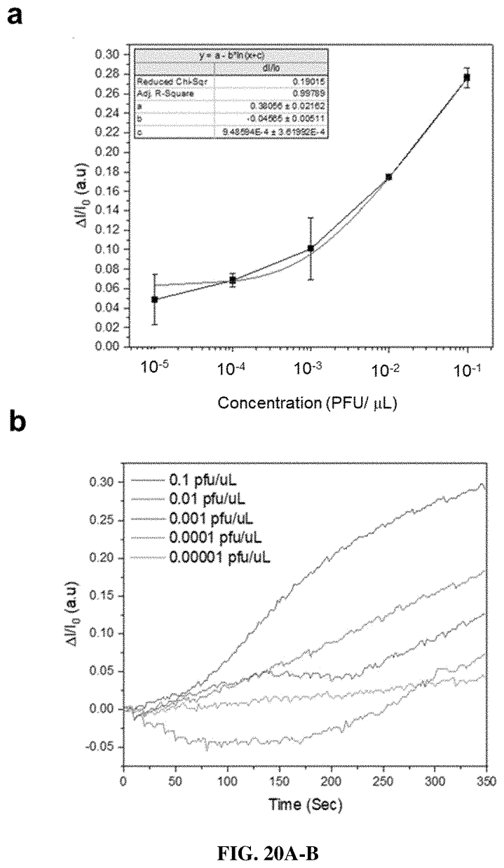

With reference to FIG. 20, a calibration curve shows the signal variation as a function of the SARS-CoV-2 virus population from 10⁻⁵ to 10⁻¹ PFU/μL. The calibration curve shows a linear relationship with the virus particle population, yielding the correlation given by ΔI/I₀=0.38+0.046 ln (C_{SARS-CoV-2}) with R²=0.99. Real-time records of the photocurrent variations were also obtained for samples with various virus particle populations. From the time change of the signal at different virus particle populations, the signal saturation generally occurred in 350 seconds. At higher populations, the noise level was low. Interestingly, at lower populations, such as C_{SARS-CoV-2=0.0001} PFU/μL, a negative photocurrent variation was rarely observed for the detection duration of 250 seconds. To prevent possible failure from water condensation in the biochip or unstable airflow, the tubing conditions are optimized by using the ⅜″ tube on the nebulizer.

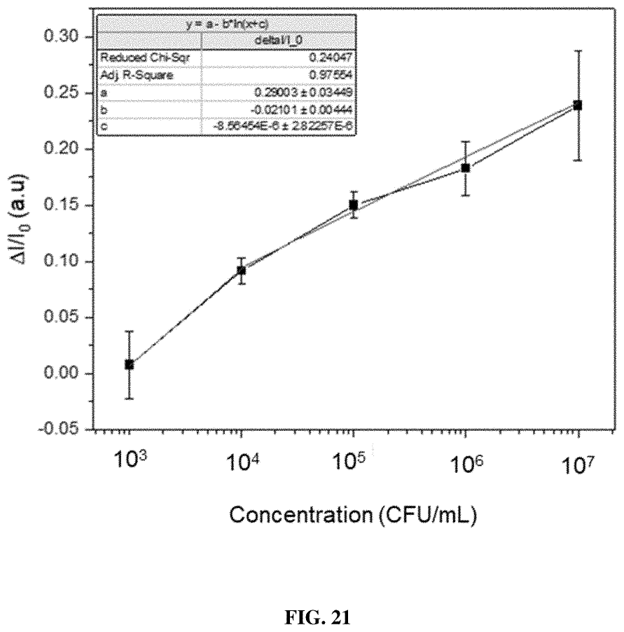

With reference to FIG. 21, the system was further tested for the detection of bacterial (E. coli) species in airflow. Measuring the average photocurrent variation as a function of the bacterial particle population results in the correlation given by ΔI/I₀=0.29+0.021 ln (C_{E. coli}) with R²=0.97. The time change of the E. coli detection signal showed that the sensor readings reached a steady state at a much faster rate. After approximately 100 seconds of detection, the photocurrent reached a balanced value with low deviation. In some embodiments, an additional calibration step is used for lowering the noise level. Each of the three detection channels are individually tested using inactivated SARS-CoV-2 samples at a population of 0.1 PFU/μL and confirmed consistent readings among all of these channels. In addition, the three detection channels reached a similar final value after 350 seconds of detection, and the photocurrent variation terms gradually increased from 0 to 0.20, which is slightly lower than the readings from the single-channel test.

As detailed herein, the integrated air quality monitoring system provides for rapid and sensitive aerosol collection and detection. The collection mechanism is based on the Venturi effect, generating negative pressure that drags air flow containing aerosols into the biochips for detection. In some embodiments, the detection involves the use of AuNPs conjugated with an antibody against SARS-CoV-2 as a nanoprobe. Experiments validate the system is capable of quantifying virus particles in the surrounding air by measuring the real-time optical transmission changes of the biofunctional AuNP-embedded hydrogel layer. Furthermore, in some embodiments, an Android IoT application permits users to control the device through Bluetooth connection and obtain real-time detection feedback from a plot on the monitoring display. The system successfully achieves the real-time and sensitive detection of SARS-CoV-2 aerosols with a dynamic concentration range of 10⁻⁵˜10⁻¹ PFU/μL.

One skilled in the art will readily appreciate that the present disclosure is well adapted to carry out the objects and obtain the ends and advantages mentioned, as well as those inherent herein. The present disclosure described herein are exemplary embodiments and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

Various features and advantages are set forth in the following claims.

What is claims is:

1.-21. (canceled)

22. A photonic biogel for spectroscopic detection of an airborne pathogen, the photonic biogel comprising:
a) a biogel comprising a cross-linked material; and
b) a plurality of plasmonic nanoprobes distributed within the biogel,
wherein the plurality of plasmonic nanoprobes are functionalized with a capture moiety that binds to an airborne pathogen.

23. The photonic biogel of claim 22, wherein the plurality of plasmonic nanoprobes are distributed substantially uniformly throughout the biogel.

24. The photonic biogel of claim 22, wherein the cross-linked material comprises a gel precursor and a cross-linking agent, wherein the ratio of the gel precursor to the cross-linking material is about 0.5 to about 2.0 (w/w).

25. The photonic biogel of claim 24, wherein the ratio of the gel precursor to the cross-linking agent is about 0.5 (w/w).

26. The photonic biogel of claim 22, wherein the plurality of plasmonic nanoprobes have an optical density within the biogel of about 0.05 to about 5.0.

27. The photonic biogel of claim 26, wherein the plurality of plasmonic nanoprobes have an optical density within the biogel of about 2.0.

28. The photonic biogel of claim 26, wherein the ratio of the gel precursor to the cross-linking agent is about 0.5 (w/w) and wherein the plurality of plasmonic nanoprobes have an optical density within the biogel of about 2.0.

29. The photonic biogel of claim 22, wherein the airborne pathogen is a virus.

30. The photonic biogel of claim 29, wherein the virus is SARS-CoV-2.

31. The photonic biogel of claim 29, wherein the capture moiety is an antibody.

32. The photonic biogel of claim 22, for use in a method of spectroscopically detecting an airborne pathogen.

33. The photonic biogel of claim 32, wherein the method of spectroscopically detecting an airborne pathogen comprises:
a) obtaining a baseline optical transmission value of the photonic biogel;
b) exposing the photonic biogel to an environment having or suspected of having the airborne pathogen; and
c) obtaining a second optical transmission value of the photonic biogel following exposure to the environment, wherein a decrease in the second optical transmission value compared to the baseline optical transmission value indicates that the airborne pathogen is present in the environment.

34. A method of spectroscopically detecting an airborne pathogen, the method comprising:
a) providing a photonic biogel, wherein the photonic biogel comprises a biogel comprising cross-linked material and a plurality of plasmonic nanoprobes distributed within the biogel, wherein the plurality of plasmonic nanoprobes are functionalized with a capture moiety that binds to the airborne pathogen;
b) obtaining a baseline optical transmission value of the photonic biogel;
c) exposing the photonic biogel to an environment having or suspected of having an airborne pathogen; and
d) obtaining a second optical transmission value of the photonic biogel following exposure to the environment,
wherein a decrease in the second optical transmission value compared to the baseline optical transmission value indicates that the airborne pathogen is present in the environment.

35. The method of claim 34, wherein the plurality of plasmonic nanoprobes are distributed substantially uniformly throughout the biogel.

36. The method of claim 34, wherein the cross-linked material comprises a gel precursor and a cross-linking agent, wherein the ratio of the gel precursor to the cross-linking material is about 0.5 to about 2.0 (w/w).

37. The method of claim 36, wherein the ratio of the gel precursor to the cross-linking agent is about 0.5 (w/w).

38. The method of claim 34, wherein the plurality of plasmonic nanoprobes have an optical density within the biogel of about 0.05 to about 5.0.

39. The method of claim 38, wherein the plurality of plasmonic nanoprobes have an optical density within the biogel of about 2.0.

40. The method of claim 36, wherein the ratio of the gel precursor to the cross-linking agent is about 0.5 (w/w) and wherein the plurality of plasmonic nanoprobes have an optical density within the biogel of about 2.0.

41. The method of claim 34, wherein the airborne pathogen is a virus.

42. The method of claim 41, wherein the capture moiety comprises an antibody.

43. The method of claim 41, wherein the virus is SARS-CoV-2.

44. The method of claim 34, wherein the airborne pathogen is a gram-negative bacteria.

45. The method of claim 44, wherein the capture moiety comprises a cysteine molecule.
